(19) United States
(12) Patent Application Publication
(10) Pub. No.: US 2015/0246888 A1
(43) Pub. Date: Sep. 3, 2015

(54) ENOYL REDUCTASE INHIBITORS WITH ANTIBACTERIAL ACTIVITY

(71) Applicants: Michael JOHNSON, Winnetka, IL (US); Shahila CHRISTIE, Bolingbrook, IL (US); Arun GHOSH, West Lafayette, IN (US); Kirk HEVENER, (US); The Board of Trustees of the University of Illinois, Urbana, IL (US); Purdue Research Foundation, West Lafayette, IN (US)

(72) Inventors: Michael Johnson, Winnetka, IL (US); Shahila Christie, Bolingbrook, IL (US); Arun Ghosh, West Lafayette, IN (US); Kirk Hevener, Boise, ID (US)

(21) Appl. No.: 14/427,603

(22) PCT Filed: Sep. 11, 2013
(86) PCT No.: PCT/US2013/059280

§ 371 Date: Mar. 11, 2015

Related U.S. Application Data
(60) Provisional application No. 61/699,653, filed on Sep. 11, 2012.

(51) Int. Cl. C07D 235/06; C07D 491/048; C07D 498/04; C07D 471/14; C07D 487/04; C07D 235/02; C07D 405/06

(52) U.S. Cl. CPC C07D 235/06; C07D 235/02; C07D 491/048; C07D 405/06; C07D 471/14; C07D 487/04; C07D 498/04

(57) ABSTRACT

Disclosed are compounds and compositions useful as antibacterials. In particular, disclosed are enoyl reductase (FabI) inhibitors, compositions comprising such compounds, processes for producing such compounds, and methods of using such compounds.

### CROSS REFERENCE TO RELATED APPLICATION

This application claims priority to U.S. Provisional Application No. 61/699,653 filed Sep. 11, 2012, the contents of which are herein incorporated by reference.

### STATEMENT OF GOVERNMENT INTEREST

This invention was made with government support under contract no. U01 A1077949, awarded by the National Institutes of Health (NIH). The government has certain rights in the invention.

### TECHNICAL FIELD

The present disclosure relates generally to antibacterial compounds and compositions, methods of using the compounds and compositions, and processes for preparing the compounds and compositions.

### BACKGROUND

Although antimicrobial drug resistance is on the rise globally, there are few drug candidates in the discovery pipeline with novel mechanisms offering a significant improvement over current antimicrobial therapies. The situation has become so urgent that the World Health Organization has declared antimicrobial resistance to be one of the three most important threats to human health.

One particularly attractive antimicrobial drug target is the bacterial fatty acid synthesis pathway (FAS-II). In bacteria, fatty acid synthesis is carried out by a series of discrete enzymes, whereas in mammals it takes place on a single, multi-enzyme complex known as FAS-I. The FAS-I complex and the FAS-II enzymes are structurally and mechanistically distinct, which strongly implies the possibility of selective antimicrobial targeting of bacterial pathogens. The NADH-dependent enzyme, enoyl-ACP reductase I (FabI), catalyzes a rate-limiting step in the FAS-II elongation cycle, and is one of the more appealing target enzymes in this pathway. The FabI enzyme is a member of the short-chain alcohol dehydrogenase/reductase (SDR) superfamily characterized by a catalytic triad of key tyrosine, lysine, and serine residues that reduce a key double bond in the enoyl substrate.

Bacillus anthracis and Francisella tularensis, the causative agents for anthrax and tularemia, respectively, are both classified as Category A agents due to their potential use in bioterrorism and biowarfare. Multi-drug resistant strains for both have either been isolated from natural sources, or can be readily selected through standard selection processes. Further, although the wild-type strains of both are variously treatable with current antibiotics, none of these antibiotics is ideal for treatment of these diseases. Similarly, increasing drug resistance in other community and hospital-acquired pathogens is an increasing public health threat. Thus, there is a clear imperative for developing new therapeutic agents against these and other organisms.

### SUMMARY

In one aspect, disclosed is a compound of formula (I),

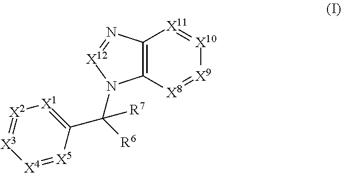

or a pharmaceutically acceptable salt, ester, amide, or prodrug form thereof,

wherein,

X¹ is selected from CR¹ and N; X² is selected from CR² and N; X³ is selected from CR³ and N; X⁴ is selected from CR⁴ and N; X⁵ is selected from CR⁵ and N; X⁸ is selected from CR⁸ and N; X⁹ is selected from CR⁹ and N; X¹⁰ is selected from CR¹⁰ and N; X¹¹ is selected from CR¹¹ and N; X¹² is selected from CR¹² and N;

R¹, R², R³, R⁴, and R⁵ are each independently selected from the group consisting of —H, —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or

R¹ and R², R² and R³, R³ and R⁴, or R⁴ and R⁵ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl;

R⁶ and R⁷ are each independently selected from the group consisting of —H, halo, and C₁-C₆-alkyl;

R⁸, R⁹, R¹⁰, and R¹¹ are each independently selected from the group consisting of —H, —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or

R⁸ and R⁹, R⁹ and R¹⁰, or R¹⁰ and R¹¹ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl; and

R¹² is selected from the group consisting of —H, —OH, —NH₂, halo, and C₁-C₆-alkyl.

In certain embodiments, X¹ is CR¹; X² is CR²; X³ is CR³; X⁴ is CR⁴; X⁵ is CR⁵; X⁸ is CR⁸; X⁹ is CR⁹; X¹⁰ is CR¹⁰; X¹¹ is CR¹¹; and X¹² is CR¹².

In certain embodiments, R⁸ is —H; and R¹¹ is —H.

In certain embodiments, R¹ is —H; and R⁵ is —H.

In certain embodiments, R¹² is —H.

In certain embodiments, R⁶ is —H; and R⁷ is —H.

In certain embodiments, R⁶ is —H; and R⁷ is methyl.

In certain embodiments, R⁴ is hydrogen.

In certain embodiments, R² and R³ are each independently selected from the group consisting of halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or R² and R³ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl.

In certain embodiments, R² and R³ are each independently selected from the group consisting of halo, C₁-C₆-alkyl, and C₁-C₆-alkoxy, or R² and R³ together with the carbon atoms to which they are attached form a substituted or unsubstituted C₃-C₆ cycloalkyl, or a substituted or unsubstituted heterocyclyl.

In certain embodiments, R² is methyl; and R³ is methoxy.

In certain embodiments, R² and R³ together with the carbon atoms to which they are attached form an unsubstituted methylenedioxy ring.

In certain embodiments, R² is chloro; and R³ is chloro.

In certain embodiments, R⁹ and R¹⁰ are each independently selected from the group consisting of C₁-C₆-alkyl, or R⁹ and R¹⁰ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl.

In certain embodiments, R⁹ is methyl; and R¹⁰ is methyl.

In certain embodiments, R⁹ and R¹⁰ together with the carbon atoms to which they are attached form a substituted or unsubstituted C₃-C₆ cycloalkyl.

In certain embodiments, R⁹ and R¹⁰ together with the carbon atoms to which they are attached form an unsubstituted cyclopentyl ring.

In certain embodiments, R⁹ and R¹⁰ together with the carbon atoms to which they are attached form an unsubstituted cyclohexyl ring.

In certain embodiments, the compound of formula (I) is selected from the group consisting of:

1-(3,4-dichlorobenzyl)-5,6-dimethyl-1H-benzo[d]imidazole;

1-(1-(3,4-dichlorophenyl)ethyl)-5,6-dimethyl-1H-benzo[d]imidazole;

1-(1-(3,4-dichlorophenyl)ethyl)-1,5,6,7-tetrahydroindeno[5,6-d]imidazole;

1-(1-(3,4-dichlorophenyl)ethyl)-5,6,7,8-tetrahydro-1H-naphtho[2,3-d]imidazole;

1-(1-(3,4-dichlorophenyl)ethyl)-6,6-dimethyl-1,5,6,7-tetrahydroindeno[5,6-d]imidazole;

1-(1-(3,4-dichlorophenyl)ethyl)-5,7-dihydro-1H-isobenzofuro[5,6-d]imidazole;

1-(4-methoxy-3-methylbenzyl)-5,6-dimethyl-1H-benzo[d]imidazole;

1-(4-methoxy-3-methylbenzyl)-1,5,6,7-tetrahydroindeno[5,6-d]imidazole;

1-(4-methoxy-3-methylbenzyl)-5,6,7,8-tetrahydro-1H-naphtho[2,3-d]imidazole;

1-(3,4-dimethoxybenzyl)-5,6,7,8-tetrahydro-1H-naphtho[2,3-d]imidazole;

1-(4-methoxy-3-methylbenzyl)-6,6-dimethyl-1,5,6,7-tetrahydroindeno[5,6-d]imidazole;

1-(3,4-dimethoxybenzyl)-6,6-dimethyl-1,5,6,7-tetrahydroindeno[5,6-d]imidazole;

1-(benzo[d][1,3]dioxol-5-ylmethyl)-6,6-dimethyl-1,5,6,7-tetrahydroindeno[5,6-d]imidazole;

1-(benzo[d][1,3]dioxol-5-ylmethyl)-5,6,7,8-tetrahydro-1H-naphtho[2,3-d]imidazole;

(S)-5-((6,6-dimethyl-6,7-dihydroindeno[5,6-d]imidazol-1(5H)-yl)methyl)-2,3-dihydro-1H-inden-1-ol;

1-(4-methoxybenzyl)-5,6-dimethyl-1H-benzo[d]imidazole;

1-(benzo[d][1,3]dioxol-5-ylmethyl)-5,6-dimethyl-1H-benzo[d]imidazole;

4-((5,6-dimethyl-1H-benzo[d]imidazol-1-yl)methyl)-2-methylphenol;

1-(benzo[d][1,3]dioxol-5-ylmethyl)-1,5,6,7-tetrahydroindeno[5,6-d]imidazole;

1-(4-methoxybenzyl)-1,5,6,7-tetrahydroindeno[5,6-d]imidazole;

4-((6,7-dihydroindeno[5,6-d]imidazol-1(5H)-yl)methyl)aniline;

1-(3,4-dimethoxybenzyl)-5,6-dimethyl-1H-benzo[d]imidazole;

1-(3,4-dimethoxybenzyl)-1,5,6,7-tetrahydroindeno[5,6-d]imidazole;

1-(4-methoxybenzyl)-5,6,7,8-tetrahydro-1H-naphtho[2,3-d]imidazole;

3-(4-(methoxymethyl)-3-methylbenzyl)-6,6-dimethyl-6,7-dihydro-3H-benzofuro[5,6-d]imidazole;

1-(4-((6,6-dimethyl-6,7-dihydro-3H-benzofuro[5,6-d]imidazol-3-yl)methyl)-2-methylphenyl)-N-methylmethanamine;

3-(4-(methoxymethyl)-3-methylbenzyl)-6,6-dimethyl-3,5,6,7-tetrahydroimidazo[4,5-f]indole;

1-(4-((6,6-dimethyl-6,7-dihydroimidazo[4,5-f]indol-3 (5H)-yl)methyl)-2-methylphenyl)-N-methylmethanamine;

5-((1-methoxy-6-methyl-2,3-dihydro-1H-inden-5-yl)methyl)-1,5-dihydroimidazo[4,5-f]indazole;

5-((1-methoxy-6-methyl-2,3-dihydro-1H-inden-5-yl)methyl)-1-methyl-1,5-dihydroimidazo[4,5-f]indazole;

(S)-3-(3-methyl-4-(tetrahydrofuran-2-yl)benzyl)-3,6,7,8-tetrahydroimidazo[4′,5′:4,5]benzo[1,2-b][1,4]oxazine;

(S)-8-methyl-3-(3-methyl-4-(tetrahydrofuran-2-yl)benzyl)-3,6,7,8-tetrahydroimidazo[4′,5′:4,5]benzo[1,2-b][1,4]oxazine;

1-(5-((7-(methoxymethyl)-5-methylimidazo[4,5-f]indol-3(5H)-yl)methyl)-2-((S)-tetrahydrofuran-2-yl)phenyl)ethanol;

1-(5-((7-(methoxymethyl)-5-methylimidazo[4,5-f]indol-3(5H)-yl)methyl)-2-((S)-tetrahydrofuran-2-yl)phenyl)-N-methylethanamine;

1-(5-((5,8-dimethyl-5,6,7,8-tetrahydro-1H-imidazo[4,5-g]quinoxalin-1-yl)methyl)indolin-1-yl)ethanone;

5,8-dimethyl-1-((1-(methylsulfonyl)indolin-5-yl)methyl)-5,6,7,8-tetrahydro-1H-imidazo[4,5-g]quinoxaline;

(S)-5-((7,8-dihydroisochromeno[6,7-d]imidazol-3(5H)-yl)methyl)-2,3-dihydro-1H-inden-2-ol;

(S)-5-((7,8-dihydroisochromeno[6,7-d]imidazol-3(5H)-yl)methyl)-N-methyl-2,3-dihydro-1H-inden-2-amine;

(R)-7-(1-(6-methyl-5,6,7,8-tetrahydro-1H-imidazo[4,5-g]isoquinolin-1-yl)ethyl)-3,4-dihydro-2H-benzo[b][1,4]oxazine;

(R)-6-((R)-1-(8-methyl-7,8-dihydroimidazo[4′,5′:4,5]benzo[1,2-b][1,4]oxazin-3(6H)-yl)ethyl)chroman-3-ol;

3-((R)-1-((R)-3-hydroxy-7-methylchroman-6-yl)ethyl)-3,6,7,8-tetrahydrothiochromeno[6,7-d]imidazole5,5-dioxide;

(S)-1-(4-methoxy-3-methylbenzyl)-1,5,6,7-tetrahydroindeno[5,6-d]imidazol-5-ol;

(S)-1-(4-methoxy-3-methylbenzyl)-1,5,6,7-tetrahydroindeno[5,6-d]imidazol-5-amine;

(S)-3-(4-methoxy-3-methylbenzyl)-3,5,6,7-tetrahydroindeno[5,6-d]imidazol-5-ol;

(S)-3-(4-methoxy-3-methylbenzyl)-3,5,6,7-tetrahydroindeno[5,6-d]imidazol-5-amine;

(5S,7S)-3-(4-methoxy-3-methylbenzyl)-7-(methylamino)-3,5,6,7-tetrahydroindeno[5,6-d]imidazol-5-ol;

(5S,7S)-7-methoxy-1-(4-methoxy-3-methylbenzyl)-N-methyl-1,5,6,7-tetrahydroindeno[5,6-d]imidazol-5-amine;

(4bR,7aR)-1-(4-methoxy-3-methylbenzyl)-1,4b,6,7,7a,8-hexahydrofuro[3,2-b]imidazo[4,5-f]indole;

(4bR,7aR)-1-(4-methoxy-3-methylbenzyl)-4b,5,6,7,7a,8-hexahydro-1H-imidazo[4,5-f]pyrrolo[3,2-b]indole;

(S)-3-(4-methoxy-3-methylbenzyl)-3,6,7,8-tetrahydrochromeno[6,7-d]imidazol-8-ol;

(S)-3-(4-methoxy-3-methylbenzyl)-5,6,7,8-tetrahydro-3H-imidazo[4,5-g]quinolin-8-ol;

(S)-3-(4-methoxy-3-methylbenzyl)-N-methyl-3,6,7,8-tetrahydrochromeno[6,7-d]imidazol-8-amine;

(S)-3-(4-methoxy-3-methylbenzyl)-N-methyl-5,6,7,8-tetrahydro-3H-imidazo[4,5-g]quinolin-8-amine;

(3aS,5S,10bS)-9-(4-methoxy-3-methylbenzyl)-1,2,3,3a,4,5,9,10b-octahydroimidazo[4′,5′:4,5]benzo[1,2-g]indo1-5-ol;

((3aR,4R,10bS)-7-(4-methoxy-3-methylbenzyl)-1,2,3,3a,4,5,7,10b-octahydroimidazo[4,5-g]pyrrolo[3,2-c]quinolin-4-yl)methanol;

(3aS,4S,10 bS)-7-(4-methoxy-3-methylbenzyl)-4-methyl-1,2,3,3a,4,5,7,10b-octahydroimidazo[4,5-g]pyrrolo[3,2-c]quinoline;

1-(4-(methoxymethyl)-3-methylbenzyl)-1,5,6,7-tetrahydroindeno[5,6-d]imidazole;

1-(4-((6,7-dihydroindeno[5,6-d]imidazol-1(5H)-yl)methyl)-2-methylphenyl)-N-methylmethanamine;

1-(4-((6,7-dihydroindeno[5,6-d]imidazol-1(5H)-yl)methyl)-2-methylphenyl)ethanol;

1-(4-((6,7-dihydroindeno[5,6-d]imidazol-1(5H)-yl)methyl)-2-methylphenyl)ethanamine;

(S)-1-(3-methyl-4-(tetrahydrofuran-2-yl)benzyl)-1,5,6,7-tetrahydroindeno[5,6-d]imidazole;

1-(5-((6,7-dihydroindeno[5,6-d]imidazol-1(5H)-yl)methyl)-2-((S)-tetrahydrofuran-2-yl)phenyl)ethanol;

1-(5-((6,7-dihydroindeno[5,6-d]imidazol-1(5H)-yl)methyl)-2-((S)-tetrahydrofuran-2-yl)phenyl)-N-methylethanamine;

1-(5-((5,6,7,8-tetrahydro-1H-naphtho[2,3-d]imidazol-1-yl)methyl)indolin-1-yl)ethanone;

1-((1-(methylsulfonyl)indolin-5-yl)methyl)-5,6,7,8-tetrahydro-1H-naphtho[2,3-d]imidazole;

(S)-5-((5,6,7,8-tetrahydro-1H-naphtho[2,3-d]imidazol-1-yl)methyl)-2,3-dihydro-1H-inden-2-ol;

(S)—N-methyl-5-((5,6,7,8-tetrahydro-1H-naphtho[2,3-d]imidazol-1-yl)methyl)-2,3-dihydro-1H-inden-2-amine;

(R)-1-(1-(1,2,3,4-tetrahydroisoquinolin-6-yl)ethyl)-5,6,7,8-tetrahydro-1H-naphtho[2,3-d]imidazole;

(R)-6-((R)-1-(5,6,7,8-tetrahydro-1H-naphtho[2,3-d]imidazol-1-yl)ethyl)chroman-3-ol;

(S)-6-amino-7-((R)-1-(5,6,7,8-tetrahydro-1H-naphtho[2,3-d]imidazol-1-yl)ethyl)-1,2,3,4-tetrahydronaphthalen-2-ol;

(S)-6-(methylamino)-7-((R)-1-(5,6,7,8-tetrahydro-1H-naphtho[2,3-d]imidazol-1-yl)ethyl)-1,2,3,4-tetrahydronaphthalen-2-ol; and

1-(4-methoxy-3-methylbenzyl)-1,5,6,7-tetrahydroindeno[5,6-d]imidazol-2-amine;

or a pharmaceutically acceptable salt, amide, ester, or prodrug form thereof.

In another aspect, disclosed is a pharmaceutical composition comprising a therapeutically effective amount of a compound of formula (I) in combination with a pharmaceutically suitable carrier. In certain embodiments, the compound of formula (I) is used for in inhibiting Enoyl-ACP Reductase (FabI). In certain embodiments, the compound of formula (I) is used for in inhibiting Enoyl-ACP Reductase (FabI) in Francisella tularensis, Staphylococcus aureus, Bacillus anthracis, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsia rickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia colt Haemophilus influenzae, Helicobacter pylori, Klebsiella pneumoniae, or Neisseria meningitidis. In certain embodiments, the compound of formula (I) is used for treating or preventing tularemia.

In another aspect, disclosed is a method for treating or preventing a bacterial infection, the method including administering to a subject in need thereof a therapeutically effective amount of a compound of formula (I). In certain embodiments, the bacterial infection is caused by a bacterium that expresses a FabI protein. In certain embodiments, the bacterial infection is caused by a bacterium selected from the group consisting of Francisella tularensis, Staphylococcus aureus, Bacillus anthracis, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsia rickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia colt Haemophilus influenzae, Helicobacter pylori, Klebsiella pneumoniae, Neisseria meningitidis, Mycobacterium tuberculosis, Plasmodium falciparum, and any combination thereof. In certain embodiments, the bacterial infection is caused by Francisella tularensis. In certain embodiments, the bacterial infection is caused by methicillin-resistant Staphylococcus aureus (MRSA), or vancomycin-resistant Staphylococcus aureus (VRSA).

The compounds, compositions, methods and processes are further described herein.

### BRIEF DESCRIPTION OF THE DRAWINGS

FIG. 1 depicts membrane depolarization at different time points upon incubation with compounds of the invention, compared to a positive control.

FIG. 2 depicts cell viability at different time points upon incubation with compounds of the invention, compared to a positive control.

### DETAILED DESCRIPTION

The present disclosure particularly relates to compounds capable of inhibiting bacterial and/or parasite fatty acid biosynthesis and their use as antibacterial and/or antiparasitic agents. Compounds of the invention may be useful as antibacterial agents having a selective spectrum of activity in vitro and in vivo against bacterial strains relying on FabI and related targets. Such strains include Francisella tularensis, Staphylococcus aureus including multiresistant strains (such as methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains), Bacillus anthracis, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsia rickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia colt Haemophilus influenzae, Helicobacter pylori, Klebsiella pneumoniae, Neisseria meningitidis and also bacteria such as Mycobacterium tuberculosis carrying homologous FabI enzymes such as InhA or other organisms such as Plasmodium falciparum.

### Definition of Terms

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

As used herein, the term “suitable substituent” is intended to mean a chemically and pharmaceutically acceptable functional group i.e., a moiety that does not negate the biological activity of the inventive compounds. Such suitable substituents may be routinely selected by those skilled in the art. Illustrative examples of suitable substituents include, but are not limited to halo groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups and the like. Those skilled in the art will appreciate that many substituents can be substituted by additional substituents.

As used herein, the term “alkenyl” refers a straight or branched hydrocarbon chain containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. Alkenyl groups of the present invention may be unsubstituted or substituted by one or more suitable substituents, preferably 1 to 3 suitable substituents, as defined above.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkoxyalkoxy” refers to an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

As used herein, the term “alkoxyalkyl” refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

As used herein, the term “alkoxycarbonyl” refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

As used herein, the term “alkyl” refers to a linear or branched hydrocarbon radical having the specified number of carbon atoms. The term “C₁-C₆-alkyl” is defined to include alkyl groups having 1, 2, 3, 4, 5, or 6 carbons in a linear or branched arrangement. For example, “C₁-C₆-alkyl” specifically includes methyl, ethyl, n-propyl, i-propyl, n-butyl, t-butyl, i-butyl, pentyl, and hexyl. Alkyl groups of the present invention may be unsubstituted or substituted by one or more suitable substituents, preferably 1 to 3 suitable substituents, as defined above.

As used herein, the term “alkylamino” refers to an alkyl group, as defined herein, appended to the parent molecular moiety through an amino group, as defined herein. Representative examples of alkylamino include, but are not limited to, methylamino, ethylamino, and sec-butylamino.

As used herein, the term “alkylaminoalkyl” refers to an alkyl group, as defined herein, appended to the parent molecular moiety through an aminoalkyl group, as defined herein. Representative examples of alkylaminoalkyl groups include, but are not limited to, methylaminoethyl and methylamino-2-propyl.

As used herein, the term “alkylcarbonyl” refers to an alkyl group appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

As used herein, the term “alkylcarbonyloxy” refers to an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

As used herein, the term “alkynyl” refers to a straight or branched hydrocarbon radical having 2, 3, 4, 5, 6, 7, 8, 9 or 10 carbons, and having one or more carbon-carbon triple bonds. Alkynyl groups of the present invention include, but are not limited to, ethynyl, propynyl, and butynyl. Alkynyl groups of the present invention may be unsubstituted or substituted by one or more suitable substituents, preferably 1 to 3 suitable substituents, as defined above.

As used herein, the term “amino” refers to an —NH₂ group.

As used herein, the term “aminoalkyl” refers to at least one amino group, as defined herein, appended to the parent molecular moiety through an alkyl group, as defined herein. Representative examples of aminoalkyl include, but are not limited to, aminomethyl, 2-aminoethyl, and 2-aminopropyl.

As used herein, the term “aryl” means monocyclic, bicyclic, or tricyclic aromatic radicals. Representative examples of the aryl groups include, but are not limited to, phenyl, dihydroindenyl, indenyl, naphthyl, dihydronaphthalenyl, and tetrahydronaphthalenyl. Aryl groups of the present invention may be optionally substituted by one or more suitable substituents, preferably 1 to 5 suitable substituents, as defined above.

As used herein, the term “carbonyl” or “(C═O)” (as used in phrases such as alkylcarbonyl, alkyl —(C═O)— or alkoxycarbonyl) refers to the joinder of the>C═O moiety to a second moiety such as an alkyl or amino group (i.e. an amido group). Alkoxycarbonylamino (i.e. alkoxy(C═O)—NH—) refers to an alkyl carbamate group. The carbonyl group is also equivalently defined herein as (C═O). Alkylcarbonylamino refers to groups such as acetamide.

As used herein, the term “cycloalkyl” refers to a mono, bicyclic or tricyclic carbocyclic radical (e.g., cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, etc.); optionally containing 1 or 2 double bonds. Cycloalkyl groups of the present invention may be unsubstituted or substituted by one or more suitable substituents, preferably 1 to 5 suitable substituents, as defined above.

As used herein, the term “di(alkyl)amino” refers to two independently selected alkyl groups, as defined herein, appended to the parent molecular moiety through an amino group, as defined herein. Representative examples of di(alkyl)amino include, but are not limited to, N,N-dimethylamino, N-ethyl-N-methylamino, and N-isopropyl-N-methylamino

As used herein, the term “di(alkyl)aminoalkyl” refers to a di(alkyl)amino group, as defined herein, appended to the parent molecular moiety through an alkyl group, as defined herein. Representative examples of di(alkyl)aminoalkyl include, but are not limited to, N,N-dimethylaminoethyl and N,N-methyl(2-propyl)aminoethyl.

As used herein, the term “halogen” or “halo” refers to a fluoro, chloro, bromo or iodo radical.

As used herein, the term “haloalkoxy” refers to an alkoxy group, as defined herein, substituted by one, two, three, or four halogen atoms. Representative examples of haloalkoxy include, but are not limited to, chloromethoxy, 2-fluoroethoxy, trifluoromethoxy, and pentafluoroethoxy.

As used herein, the term “haloalkyl” refers to an alkyl group, as defined herein, substituted by one, two, three, or four halogen atoms. Representative examples of haloalkyl include, but are not limited to, chloromethyl, 2-fluoroethyl, trifluoromethyl, pentafluoroethyl, 2-chloro-3-fluoropentyl, and 4,4,4,-trifluorobutyl.

As used herein, the term “heteroaryl” refers to a monocyclic heteroaryl or a bicyclic heteroaryl. The monocyclic heteroaryl is a five- or six-membered ring. The five-membered ring contains two double bonds. The five-membered ring may contain one heteroatom selected from O or S; or one, two, three, or four nitrogen atoms and optionally one oxygen or sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl includes a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryl groups include, but are not limited to, benzofuranyl, benzothienyl, benzoxazolyl, benzimidazolyl, benzoxadiazolyl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinazolinyl, quinolinyl, thiazolo[5,4-b]pyridin-2-yl, thiazolo[5,4-d]pyrimidin-2-yl, and 5,6,7,8-tetrahydroquinolin-5-yl. Heteroaryl groups of the present invention may be unsubstituted or substituted by one or more suitable substituents, preferably 1 to 5 suitable substituents, as defined above.

As used herein, the term “heterocycle” or “heterocyclyl” refers to a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of oxygen, nitrogen, phosphorus and sulfur. The three- or four-membered ring contains zero or one double bond, and one heteroatom selected from the group consisting of oxygen, nitrogen, phosphorus and sulfur. The five-membered ring contains zero or one double bond and one, two or three heteroatoms selected from the group consisting of oxygen, nitrogen, phosphorus and sulfur. The six-membered ring contains zero, one or two double bonds and one, two, or three heteroatoms selected from the group consisting of oxygen, nitrogen, phosphorus and sulfur. The seven- and eight-membered rings contains zero, one, two, or three double bonds and one, two, or three heteroatoms selected from the group consisting of oxygen, nitrogen, phosphorus and sulfur. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, phosphinane, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydropyrimidinyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, 9-phosphabicyclo[3.3.1]nonane, 8-phosphabicyclo[3.2.1]octane, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-admantane (1-azatricyclo[3.3.1.1^{3,7}]decane), oxa-adamantane (2-oxatricyclo[3.3.1.1^{3,7}]decane), and 2,4,6-trioxa-8-phosphatricyclo[3.3.1.13,7]decane. Heterocyclic groups of the present invention may be unsubstituted or substituted by one or more suitable substituents, preferably 1 to 3 suitable substituents, as defined above.

As used herein, the term “hydroxy” refers to an —OH group.

As used herein, the term “hydroxyalkoxy” refers to an alkoxy group, as defined herein, substituted by at least one hydroxy group. Representative examples of hydroxyalkoxy include, but are not limited to, hydroxyethoxy, and 2-hydroxypropoxy.

As used herein, the term “hydroxyalkyl” refers to an alkyl group, as defined herein, substituted by at least one hydroxy group. Representative examples of hydroxyalkyl include, but are not limited to, hydroxymethyl, 2-hydroxyethyl, 3-hydroxypropyl, 2,3-dihydroxypropyl, 2,3-dihydroxypentyl, 4-hydroxybutyl, 2-ethyl-4-hydroxyheptyl, 3,4-dihydroxybutyl, and 5-hydroxypentyl.

As used herein, the term “methylenedioxy” refers to a —OCH₂O— group wherein the oxygen atoms of the methylenedioxy are attached to the parent molecular moiety through two adjacent carbon atoms.

As used herein, the term “nitrogen protecting group” refers to groups intended to protect an amino group against undesirable reactions during synthetic procedures. Representative nitrogen protecting groups include acetyl, benzoyl, benzyl, benzyloxycarbonyl (Cbz), formyl, phenylsulfonyl, tert-butoxycarbonyl (Boc), tert-butylacetyl, trifluoroacetyl, and triphenylmethyl (trityl).

As used herein, the term “oxo” refers to a double bonded oxygen (═O) radical wherein the bond partner is a carbon atom. Such a radical can also be thought as a carbonyl group.

A prefix attached to a multi-component substituent only applies to the first component it precedes. To illustrate, the term “alkylcycloalkyl” contains two components: alkyl and cycloalkyl. Thus, the C₁-C₆-prefix on C₁-C₆-alkylcycloalkyl means that the alkyl component of the alkylcycloalkyl contains from 1 to 6 carbon atoms; the C₁-C₆-prefix does not describe the cycloalkyl component. To illustrate further, the prefix “halo” on haloalkoxyalkyl indicates that only the alkoxy component of the alkoxyalkyl substituent is substituted with one or more halogen radicals. If the halogen substitution may only occur on the alkyl component, the substituent would instead be described as “alkoxyhaloalkyl.”

A substituent is “substitutable” if it comprises at least one carbon or nitrogen atom that is bonded to one or more hydrogen atoms. Thus, for example, hydrogen, halogen, and cyano do not fall within this definition. In addition, a sulfur atom in a heterocyclyl containing such atom is substitutable with one or two oxo substituents.

If a substituent is described as being “substituted”, a non-hydrogen radical is in the place of hydrogen radical on a carbon or nitrogen of the substituent. Thus, for example, a substituted alkyl substituent is an alkyl substituent in which at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there is more than one substitution on a substituent, each non-hydrogen radical may be identical or different (unless otherwise stated).

When a substituent is referred to as “unsubstituted” or not referred to as “substituted” or “optionally substituted”, it means that the substituent does not have any substituents. If a substituent is described as being “optionally substituted”, the substituent may be either (1) not substituted or (2) substituted. If a substituent is described as being optionally substituted with up to a particular number of non-hydrogen radicals, that substituent may be either (1) not substituted; or (2) substituted by up to that particular number of non-hydrogen radicals or by up to the maximum number of substitutable positions on the substituent, whichever is less. Thus, for example, if a substituent is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

If substituents are described as being “independently selected” from a group, each substituent is selected independent of the other. Each substituent, therefore, may be identical to or different from the other substituent(s).

### Compounds

In one aspect, compounds of the invention have formula (I),

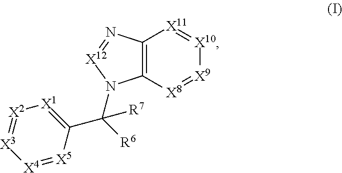

or a pharmaceutically acceptable salt, ester, amide, or prodrug form thereof,

wherein

X¹ is selected from CR¹ and N; X² is selected from CR² and N; X³ is selected from CR³ and N; X⁴ is selected from CR⁴ and N; X⁵ is selected from CR⁵ and N; X⁸ is selected from CR⁸ and N; X⁹ is selected from CR⁹ and N; X¹⁰ is selected from CR¹⁰ and N; X¹¹ is selected from CR¹¹ and N; X¹² is selected from CR¹² and N;

R¹, R², R³, R⁴, and R⁵ are each independently selected from the group consisting of —H, —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-S0₂-, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or

R¹ and R², R² and R³, R³ and R⁴, or R⁴ and R⁵ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl;

R⁶ and R⁷ are each independently selected from the group consisting of —H, halo, and C₁-C₆-alkyl;

R⁸, R⁹, R¹⁰, and R¹¹ are each independently selected from the group consisting of —H, —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or

R⁸ and R⁹, R⁹ and R¹⁰, or R¹⁰ and R¹¹ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl; and

R¹² is selected from the group consisting of —H, —OH, —NH₂, halo, and C₁-C₆-alkyl.

In certain embodiments, said heteroaryl is, at each occurrence, independently selected from a five- or six-membered heteroaryl having one, two, three, or four heteroatoms independently selected from nitrogen, oxygen, and sulfur.

In certain embodiments, said heterocyclyl is, at each occurrence, independently selected from a five- or six-membered heterocyclyl containing one, two, or three heteroatoms independently selected from nitrogen, oxygen, and sulfur.

In certain embodiments, said aryl is a C₆-C₁₂-aryl. In certain embodiments, said aryl is a substituted or unsubstituted phenyl group. In certain embodiments, said aryl is an unsubstituted phenyl.

In certain embodiments, said C₃-C₆ cycloalkyl, heteroaryl, heterocyclyl, and aryl, are each independently optionally substituted with 1 to 3 substituents independently selected from —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, and C₂-C₆-alkynyl, or two adjacent atoms of said C₃-C₆ cycloalkyl, heteroaryl, heterocyclyl, or aryl, together with two, three, or four atoms independently selected from carbon, nitrogen, oxygen, and sulfur, form a substituted or unsubstituted four-, five-, or six-membered cycloalkyl or heterocyclyl or five- or six-membered aryl or heteroaryl.

In certain embodiments, X¹ is CR¹; X² is CR²; X³ is CR³; X⁴ is CR⁴; X⁵ is CR⁵; X⁸ is CR⁸; X⁹ is CR⁹; X¹⁰ is CR¹⁰; X¹¹ is CR¹¹; and X¹² is CR¹². In certain embodiments, one of X¹, X², X³, X⁴, and X⁵ is N. In certain embodiments, two of X¹, X², X³, X⁴, and X⁵ are N. In certain embodiments, three of X¹, X², X³, X⁴, and X⁵ are N. In certain embodiments, four of X¹, X², X³, X⁴, and X⁵ are N. In certain embodiments, one of X⁸, X⁹, X¹⁰, X¹¹ is N. In certain embodiments, two of X8, X⁹, X¹⁰, X¹¹ are N. In certain embodiments, three of X⁸, X⁹, X¹⁰, X¹¹ are N. In certain embodiments, four of X⁸, X⁹, X¹⁰, X¹¹ are N.

In certain embodiments, X¹ is CR¹; X² is CR²; X³ is CR³; X⁴ is CR⁴; X⁵ is CR⁵; X⁸ is CR⁸; X⁹ is CR⁹; X¹⁰ is CR¹⁰; X¹¹ is CR¹¹; and X¹² is CR¹²; wherein R¹, R⁴, R⁵, R⁶, R⁸, R¹¹, and R¹² are each hydrogen; wherein R⁷ is hydrogen or methyl; wherein R² and R³ are each independently selected from the group consisting of —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or R² and R³ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl; wherein R⁹ and R¹⁰ are each independently selected from the group consisting of —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or R² and R³ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl.

In certain embodiments, X¹ is CR¹; X² is CR²; X³ is CR³; X⁴ is CR⁴; X⁵ is CR⁵; X⁸ is CR⁸; X⁹ is CR⁹; X¹⁰ is CR¹⁰; X¹¹ is CR¹¹; and X¹² is CR¹²; wherein R¹, R⁴, R⁵, R⁶, R⁸, R¹¹, and R¹² are each hydrogen; wherein R⁷ is hydrogen or methyl; wherein R² and R³ are each independently selected from the group consisting of halo, C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, and substituted or unsubstituted heterocyclyl, or R² and R³ together with the carbon atoms to which they are attached form a substituted or unsubstituted C₅-C₆ cycloalkyl, or a substituted or unsubstituted heterocyclyl; wherein R⁹ and R¹⁰ are each independently selected from the group consisting of C₁-C₆-alkyl, or R⁹ and R¹⁰ together with the carbon atoms to which they are attached form a substituted or unsubstituted C₅-C₆ cycloalkyl, a substituted or unsubstituted heteroaryl, or a substituted or unsubstituted heterocyclyl.

In certain embodiments, X¹ is CR¹; X² is CR²; X³ is CR³; X⁴ is CR⁴; X⁵ is CR⁵; X⁸ is CR⁸; X⁹ is CR⁹; X¹⁰ is CR¹⁰; X¹¹ is CR¹¹; and X¹² is CR¹²; wherein R¹, R⁴, R⁵, R⁶, R⁸, R¹¹, and R¹² are each hydrogen; wherein R⁷ is hydrogen or methyl; wherein R² and R³ are each independently selected from the group consisting of chloro, methyl, hydroxyethyl, aminoethyl, methylaminomethyl, methoxymethyl, methoxy, and unsubstituted tetrahydrofuranyl, or R² and R³ together with the carbon atoms to which they are attached form a substituted or unsubstituted C₅-C₆ cycloalkyl, or a substituted or unsubstituted 5- or 6-membered heterocyclyl; wherein R⁹ and R¹⁰ are each methyl, or R⁹ and R¹⁰ together with the carbon atoms to which they are attached form a substituted or unsubstituted C₅-C₆ cycloalkyl, a substituted or unsubstituted heteroaryl, or a substituted or unsubstituted 5- or 6-membered heterocyclyl.

In certain embodiments, R² is C₁-C₆-alkyl; and R³ is C₁-C₆-alkoxy. In certain embodiments, R² is methyl; and R³ is methoxy.

In certain embodiments, R² and R³ together with the carbon atoms to which they are attached form a C₅-C₆ cycloalkyl optionally substituted by 1 to 3 substituents independently selected from —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, and C₂-C₆-alkynyl.

In certain embodiments, R² and R³ together with the carbon atoms to which they are attached form a C₅-C₆ cycloalkyl optionally substituted by 1 substituent selected from —OH, C₁-C₆-alkoxy, and C₁-C₆-alkylamino.

In certain embodiments, R² and R³ together with the carbon atoms to which they are attached form a C₅-C₆ cycloalkyl optionally substituted by 1 substituent selected from —OH, methoxy, and methylamino.

In certain embodiments, R² and R³ together with the carbon atoms to which they are attached form a 5- or 6-membered heterocyclyl containing 1 to 3 heteroatoms independently selected from nitrogen, oxygen, and sulfur, said hetercyclyl optionally substituted by 1 to 3 substituents independently selected from —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, and C₂-C₆-alkynyl.

In certain embodiments, R² and R³ together with the carbon atoms to which they are attached form a 5- or 6-membered heterocyclyl selected from:

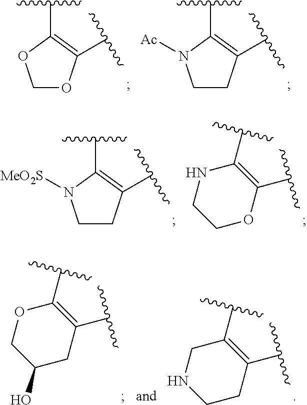

In certain embodiments, R⁹ and R¹⁰ together with the carbon atoms to which they are attached form a C₅-C₆ cycloalkyl optionally substituted by 1 to 3 substituents independently selected from —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, and C₂-C₆-alkynyl, or two adjacent carbon atoms of said C₅-C₆ cycloalkyl, together with two, three, or four atoms independently selected from carbon, nitrogen, oxygen, and sulfur, form a substituted or unsubstituted four-, five-, or six-membered cycloalkyl or heterocyclyl.

In certain embodiments, R⁹ and R¹⁰ together with the carbon atoms to which they are attached form a C₅-C₆ cycloalkyl optionally substituted by 1 to 3 substituents independently selected from —OH, —NH₂, C₁-C₆-alkyl, C₁-C₆-alkoxy, and C₁-C₆-alkylamino, or two adjacent carbon atoms of said C₅-C₆ cycloalkyl, together with two carbon atoms and one nitrogen atom, form a five-membered heterocyclyl.

In certain embodiments, R⁹ and R¹⁰ together with the carbon atoms to which they are attached form a C₅-C₆ cycloalkyl optionally substituted by 1 to 3 substituents independently selected from —OH, —NH₂, methyl, methoxy, and methylamino, or two adjacent carbon atoms of said C₅-C₆ cycloalkyl, together with two carbon atoms and one nitrogen atom, form an unsubstituted pyrrolidinyl.

In certain embodiments, R⁹ and R¹⁰ together with the carbon atoms to which they are attached form a substituted or unsubstituted C₅-C₆ cycloalkyl selected from:

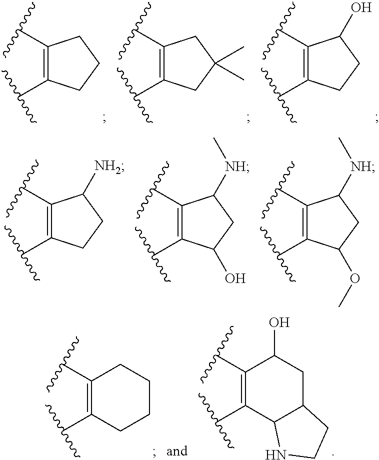

In certain embodiments, R⁹ and R¹⁰ together with the carbon atoms to which they are attached form a 5- or 6-membered heterocyclyl containing 1 to 3 heteroatoms independently selected from nitrogen, oxygen, and sulfur, said hetercyclyl optionally substituted by 1 to 3 substituents independently selected from —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, and C₂-C₆-alkynyl, or two adjacent atoms of said heterocyclyl, together with two, three, or four atoms independently selected from carbon, nitrogen, oxygen, and sulfur, form a substituted or unsubstituted four-, five-, or six-membered cycloalkyl or heterocyclyl.

In certain embodiments, R⁹ and R¹⁰ together with the carbon atoms to which they are attached form a 5- or 6-membered heterocyclyl containing 1 to 3 heteroatoms independently selected from nitrogen, oxygen, and sulfur, said hetercyclyl optionally substituted by 1 to 3 substituents independently selected from —OH, —NH₂, C₁-C₆-alkyl, hydroxy-C₁-C₆-alkyl, C₁-C₆-alkoxy-C₁-C₆-alkyl, C₁-C₆-alkoxy, and C₁-C₆-alkylamino, or two adjacent atoms of said heterocyclyl, together with three, or four atoms independently selected from carbon, nitrogen, oxygen, and sulfur, form a substituted or unsubstituted five-, or six-membered heterocyclyl.

In certain embodiments, R⁹ and R¹⁰ together with the carbon atoms to which they are attached form a 5- or 6-membered heterocyclyl selected from:

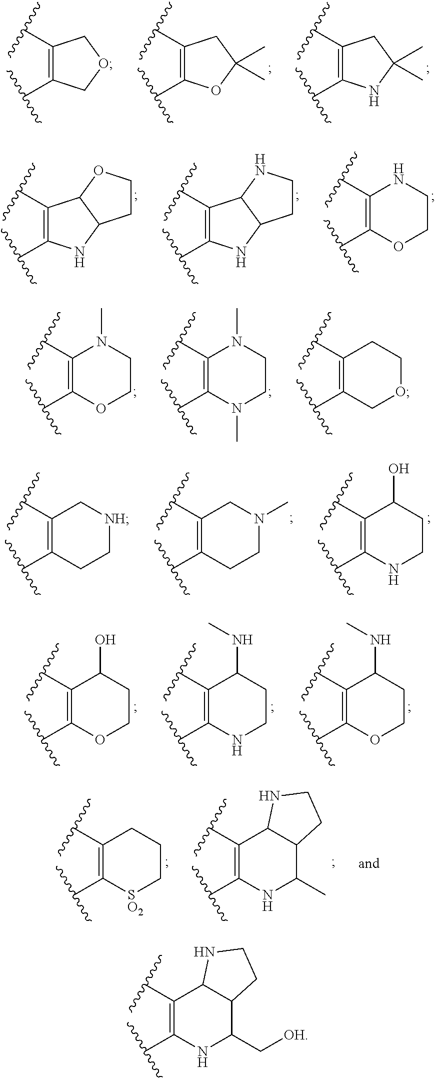

In certain embodiments, R⁹ and R¹⁰ together with the carbon atoms to which they are attached form a 5- or 6-membered heteroaryl containing 1 to 3 heteroatoms independently selected from nitrogen, oxygen, and sulfur, said heteroaryl optionally substituted by 1 to 3 substituents independently selected from —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, and C₂-C₆-alkynyl.

In certain embodiments, R⁹ and R¹⁰ together with the carbon atoms to which they are attached form a 5- or 6-membered heteroaryl containing 1 to 3 heteroatoms independently selected from nitrogen, oxygen, and sulfur, said heteroaryl optionally substituted by 1 to 3 substituents independently selected from —OH, —NH₂, C₁-C₆-alkyl, hydroxy-C₁-C₆-alkyl, C₁-C₆-alkoxy-C₁-C₆-alkyl, C₁-C₆-alkoxy, and C₁-C₆-alkylamino.

In certain embodiments, R⁹ and R¹⁰ together with the carbon atoms to which they are attached form a 5- or 6-membered heteroaryl selected from:

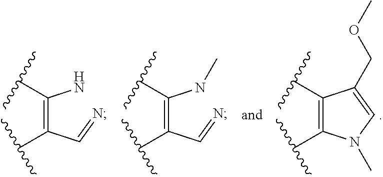

In certain embodiments, compounds of the invention have formula (I-a),

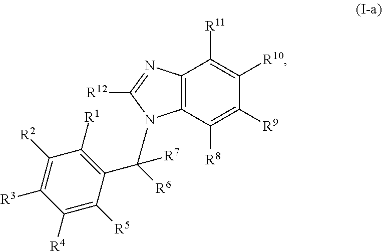

wherein

R¹, R², R³, R⁴, R⁵, R⁶, R⁷, R⁸, R⁹, R¹⁰, R¹¹, and R¹² are as defined above.

In certain embodiments, compounds of the invention have formula (I-b),

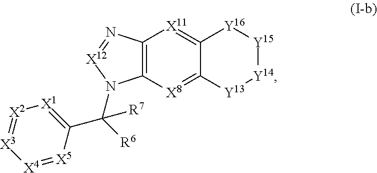

wherein

Y¹³ is selected from C(R^{13a}R^{13b}), NR^{13c}, O, and S, or Y¹³ is a bond;

Y¹⁴ is selected from C(R^{14a}R^{14b}), NR^{14c}, O, and S, or Y¹⁴ is a bond;

Y¹⁵ is selected from C(R^{15a}R^{15b}), NR^{15c}, O, and S, or Y¹⁵ is a bond;

Y¹⁶ is selected from C(R^{16a}R^{16b}), NR^{16c}, O, and S, or Y¹⁶ is a bond;

provided that no more than one of Y¹³, Y¹⁴, Y¹⁵, and Y¹⁶ is a bond;

R^{13a}, R^{13b}, R^{14a}, R^{14b}, R^{15a}, R^{15b}, R^{16a}, and R ^{16b} are each independently selected from the group consisting of —H, —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl;

R^{13c}, R^{14c}, R^{15c}, R^{16c} are each independently selected from the group consisting of —H and C₁-C₆-alkyl; and

X¹, X², X³, X⁴, X⁵, X⁸, X¹¹, X¹², R⁶, and R⁷ are as defined above.

In certain embodiments, compounds of the invention have formula (I-c),

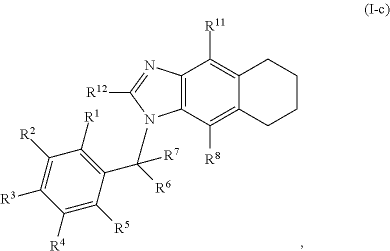

wherein R¹, R², R³, R⁴, R⁵, R⁶, R⁷, R⁸, R¹¹, and R¹² are as defined above.

In certain embodiments, compounds of the invention have formula (I-d),

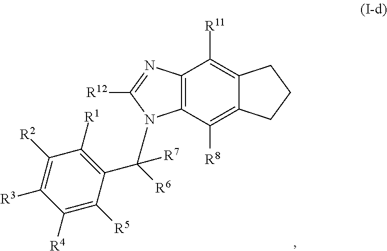

wherein R¹, R², R³, R⁴, R⁵, R⁶, R⁷, R⁸, R¹¹, and R¹² are as defined above.

In certain embodiments, compounds of the invention have formula (I-e),

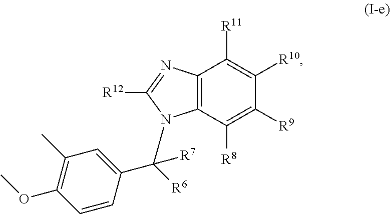

wherein R⁶, R⁷, R8, _{R}9_{, R}¹⁰, R¹¹, and R¹² are as defined above.

In certain embodiments, compounds of the invention have formula (I-f),

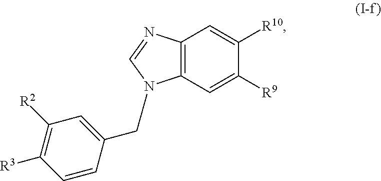

wherein R², R³, R⁹, and R¹⁰ are as defined above.

In certain embodiments, with respect to compounds of formula (I-f), R², R³, R⁹, and R¹⁰ are each independently selected from the group consisting of —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or

R² and R³ together with the carbon atoms to which they are attached form a substituted or unsubstituted C₅-C₆ cycloalkyl, a substituted or unsubstituted heteroaryl, a substituted or unsubstituted heterocyclyl, or a substituted or unsubstituted aryl, or

R⁹ and R¹⁰ together with the carbon atoms to which they are attached form a substituted or unsubstituted C₅-C₆ cycloalkyl, a substituted or unsubstituted heteroaryl, a substituted or unsubstituted heterocyclyl, or a substituted or unsubstituted aryl.

In certain embodiments, with respect to compounds of formula (I-f), R² is methyl; and R³ is methoxy. In certain embodiments, with respect to compounds of formula (I-f), R² is methyl; R³ is methoxy; and R⁹ and R¹⁰ together with the carbon atoms to which they are attached form a substituted or unsubstituted C₅-C₆ cycloalkyl, or a substituted or unsubstituted heterocyclyl. In certain embodiments, with respect to compounds of formula (I-f), R⁹ and R¹⁰ together with the carbon atoms to which they are attached form a substituted or unsubstituted C₅-C₆ cycloalkyl, or a substituted or unsubstituted heterocyclyl.

In certain embodiments, compounds of the invention have formula (I-g),

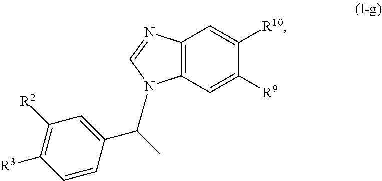

wherein R², R³, R⁹, and R¹⁰ are as defined above.

In certain embodiments, with respect to compounds of formula (I-g), R², R³, R⁹, and R¹⁰ are each independently selected from the group consisting of —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or

R² and R³ together with the carbon atoms to which they are attached form a substituted or unsubstituted C₅-C₆ cycloalkyl, a substituted or unsubstituted heteroaryl, a substituted or unsubstituted heterocyclyl, or a substituted or unsubstituted aryl, or

R⁹ and R¹⁰ together with the carbon atoms to which they are attached form a substituted or unsubstituted C₅-C₆ cycloalkyl, a substituted or unsubstituted heteroaryl, a substituted or unsubstituted heterocyclyl, or a substituted or unsubstituted aryl.

In certain embodiments, with respect to compounds of formula (I-g), R² is methyl; and R³ is methoxy. In certain embodiments, with respect to compounds of formula (I-g), R² is methyl; R³ is methoxy; and R⁹ and R¹⁰ together with the carbon atoms to which they are attached form a substituted or unsubstituted C₅-C₆ cycloalkyl, or a substituted or unsubstituted heterocyclyl. In certain embodiments, with respect to compounds of formula (I-g), R⁹ and R¹⁰ together with the carbon atoms to which they are attached form a substituted or unsubstituted C₅-C₆ cycloalkyl, or a substituted or unsubstituted heterocyclyl.

Exemplary compounds of the invention include, but are not limited to,

1-(3,4-dichlorobenzyl)-5,6-dimethyl-1H-benzo[d]imidazole;

1-(1-(3,4-dichlorophenyl)ethyl)-5,6-dimethyl-1H-benzo[d]imidazole;

1-(1-(3,4-dichlorophenyl)ethyl)-1,5,6,7-tetrahydroindeno[5,6-d]imidazole;

1-(1-(3,4-dichlorophenyl)ethyl)-5,6,7,8-tetrahydro-1H-naphtho[2,3-d]imidazole;

1-(1-(3,4-dichlorophenyl)ethyl)-6,6-dimethyl-1,5,6,7-tetrahydroindeno[5,6-d]imidazole;

1-(1-(3,4-dichlorophenyl)ethyl)-5,7-dihydro-1H-isobenzofuro[5,6-d]imidazole;

1-(4-methoxy-3-methylbenzyl)-5,6-dimethyl-1H-benzo[d]imidazole;

1-(4-methoxy-3-methylbenzyl)-1,5,6,7-tetrahydroindeno[5,6-d]imidazole;

1-(4-methoxy-3-methylbenzyl)-5,6,7,8-tetrahydro-1H-naphtho[2,3-d]imidazole;

1-(3,4-dimethoxybenzyl)-5,6,7,8-tetrahydro-1H-naphtho[2,3-d]imidazole;

1-(4-methoxy-3-methylbenzyl)-6,6-dimethyl-1,5,6,7-tetrahydroindeno[5,6-d]imidazole;

1-(3,4-dimethoxybenzyl)-6,6-dimethyl-1,5,6,7-tetrahydroindeno[5,6-d]imidazole;

1-(benzo[d][1,3]dioxol-5-ylmethyl)-6,6-dimethyl-1,5,6,7-tetrahydroindeno[5,6-d]imidazole;

1-(benzo[d][1,3]dioxol-5-ylmethyl)-5,6,7,8-tetrahydro-1H-naphtho[2,3-d]imidazole;

(S)-5-((6,6-dimethyl-6,7-dihydroindeno[5,6-d]imidazol-1(5H)-yl)methyl)-2,3-dihydro-1H-inden-1-ol;

1-(4-methoxybenzyl)-5,6-dimethyl-1H-benzo[d]imidazole;

1-(benzo[d][1,3]dioxol-5-ylmethyl)-5,6-dimethyl-1H-benzo[d]imidazole;

4-((5,6-dimethyl-1H-benzo[d]imidazol-1-yl)methyl)-2-methylphenol;

1-(benzo[d][1,3]dioxol-5-ylmethyl)-1,5,6,7-tetrahydroindeno[5,6-d]imidazole;

1-(4-methoxybenzyl)-1,5,6,7-tetrahydroindeno[5,6-d]imidazole;

4-((6,7-dihydroindeno[5,6-d]imidazol-1(5H)-yl)methyl)aniline;

1-(3,4-dimethoxybenzyl)-5,6-dimethyl-1H-benzo[d]imidazole;

1-(3,4-dimethoxybenzyl)-1,5,6,7-tetrahydroindeno[5,6-d]imidazole;

1-(4-methoxybenzyl)-5,6,7,8-tetrahydro-1H-naphtho[2,3-d]imidazole;

3-(4-(methoxymethyl)-3-methylbenzyl)-6,6-dimethyl-6,7-dihydro-3H-benzofuro[5,6-d]imidazole;

1-(4-((6,6-dimethyl-6,7-dihydro-3H-benzofuro[5,6-d]imidazol-3-yl)methyl)-2-methylphenyl)-N-methylmethanamine;

3-(4-(methoxymethyl)-3-methylbenzyl)-6,6-dimethyl-3,5,6,7-tetrahydroimidazo[4,5-f]indole;

1-(4-((6,6-dimethyl-6,7-dihydroimidazo[4,5-f]indol-3(5H)-yl)methyl)-2-methylphenyl)-N-methylmethanamine;

5-((1-methoxy-6-methyl-2,3-dihydro-1H-inden-5-yl)methyl)-1,5-dihydroimidazo[4,5-f]indazole;

5-((1-methoxy-6-methyl-2,3-dihydro-1H-inden-5-yl)methyl)-1-methyl-1,5-dihydroimidazo[4,5-f]indazole;

(S)-3-(3-methyl-4-(tetrahydrofuran-2-yl)benzyl)-3,6,7,8-tetrahydroimidazo[4′,5′:4,5]benzo[1,2-b][1,4]oxazine;

(S)-8-methyl-3-(3-methyl-4-(tetrahydrofuran-2-yl)benzyl)-3,6,7,8-tetrahydroimidazo[4′,5′:4,5]benzo[1,2-b][1,4]oxazine;

1-(5-((7-(methoxymethyl)-5-methylimidazo[4,5-f]indol-3(5H)-yl)methyl)-2-((S)-tetrahydrofuran-2-yl)phenyl)ethanol;

1-(5-((7-(methoxymethyl)-5-methylimidazo[4,5-f]indol-3(5H)-yl)methyl)-2-((S)-tetrahydrofuran-2-yl)phenyl)-N-methylethanamine;

1-(5-((5,8-dimethyl-5,6,7,8-tetrahydro-1H-imidazo[4,5-g]quinoxalin-1-yl)methyl)indolin-1-yl)ethanone;

5,8-dimethyl-1-((1-(methylsulfonyl)indolin-5-yl)methyl)-5,6,7,8-tetrahydro-1H-imidazo[4,5-g]quinoxaline;

(S)-5-((7,8-dihydroisochromeno[6,7-d]imidazol-3(5H)-yl)methyl)-2,3-dihydro-1H-inden-2-ol;

(S)-5-(7,8-dihydroisochromeno[6,7-d]imidazol-3(5H)-yl)methyl)-N-methyl-2,3-dihydro-1H-inden-2-amine;

(R)-7-(1-(6-methyl-5,6,7,8-tetrahydro-1H-imidazo[4,5-g]isoquinolin-1-yl)ethyl)-3,4-dihydro-2H-benzo[b][1,4]oxazine;

(R)-6-((R)-1-(8-methyl-7,8-dihydroimidazo[4′,5′:4,5 ]benzo[1,2-b][1,4]oxazin-3(6H)-yl)ethyl)chroman-3-ol;

3-((R)-1-((R)-3-hydroxy-7-methylchroman-6-yl)ethyl)-3,6,7,8-tetrahydrothiochromeno[6,7-d]imidazole 5,5-dioxide;

(S)-1-(4-methoxy-3-methylbenzyl)-1,5,6,7-tetrahydroindeno[5,6-d]imidazol-5-ol;

(S)-1-(4-methoxy-3-methylbenzyl)-1,5,6,7-tetrahydroindeno[5,6-d]imidazol-5-amine;

(S)-3-(4-methoxy-3-methylbenzyl)-3,5,6,7-tetrahydroindeno[5,6-d]imidazol-5-ol;

(S)-3-(4-methoxy-3-methylbenzyl)-3,5,6,7-tetrahydroindeno[5,6-d]imidazol-5-amine;

(5S,7S)-3-(4-methoxy-3-methylbenzyl)-7-(methylamino)-3,5,6,7-tetrahydroindeno[5,6-d]imidazol-5-ol;

(5S,7S)-7-methoxy-1-(4-methoxy-3-methylbenzyl)-N-methyl-1,5,6,7-tetrahydroindeno[5,6-d]imidazol-5-amine;

(4bR,7aR)-1-(4-methoxy-3-methylbenzyl)-1,4b,6,7,7a,8-hexahydrofuro[3,2-b]imidazo[4,5-f]indole;

(4bR,7aR)-1-(4-methoxy-3-methylbenzyl)-4b,5,6,7,7a,8-hexahydro-1H-imidazo[4,5-f]pyrrolo[3,2-b]indole;

(S)-3-(4-methoxy-3-methylbenzyl)-3,6,7,8-tetrahydrochromeno[6,7-d]imidazol-8-ol;

(S)-3-(4-methoxy-3-methylbenzyl)-5,6,7,8-tetrahydro-3H-imidazo[4,5-g]quinolin-8-ol;

(S)-3-(4-methoxy-3-methylbenzyl)-N-methyl-3,6,7,8-tetrahydrochromeno[6,7-d]imidazol-8-amine;

(S)-3-(4-methoxy-3-methylbenzyl)-N-methyl-5,6,7,8-tetrahydro-3H-imidazo[4,5-g]quinolin-8-amine;

(3aS,5S,10bS)-9-(4-methoxy-3-methylbenzyl)-1,2,3,3a,4,5,9,10b-octahydroimidazo[4′,5′:4,5]benzo[1,2-g]indol-5-ol;

((3aR,4R,10bS)-7-(4-methoxy-3-methylbenzyl)-1,2,3,3a,4,5,7,10b-octahydroimidazo[4,5-g]pyrrolo[3,2-c]quinolin-4-yl)methanol;

(3aS,4S,10bS)-7-(4-methoxy-3-methylbenzyl)-4-methyl-1,2,3,3a,4,5,7,10b-octahydroimidazo[4,5-g]pyrrolo[3,2-c]quinoline;

1-(4-(methoxymethyl)-3-methylbenzyl)-1,5,6,7-tetrahydroindeno[5,6-d]imidazole;

1-(4-((6,7-dihydroindeno[5,6-d]imidazol-1(5H)-yl)methyl)-2-methylphenyl)-N-methylmethanamine;

1-(4-((6,7-dihydroindeno[5,6-d]imidazol-1(5H)-yl)methyl)-2-methylphenyl)ethanol;

1-(4-((6,7-dihydroindeno[5,6-d]imidazol-1(5H)-yl)methyl)-2-methylphenyl)ethanamine;

(S)-1-(3-methyl-4-(tetrahydrofuran-2-yl)benzyl)-1,5,6,7-tetrahydroindeno[5,6-d]imidazole;

1-(5-((6,7-dihydroindeno[5,6-d]imidazol-1(5H)-yl)methyl)-2-((S)-tetrahydrofuran-2-yl)phenyl)ethanol;

1-(5-((6,7-dihydroindeno[5,6-d]imidazol-1(5H)-yl)methyl)-2-((S)-tetrahydrofuran-2-yl)phenyl)-N-methylethanamine;

1-(5-((5,6,7,8-tetrahydro-1H-naphtho[2,3-d]imidazol-1-yl)methyl)indolin-1-yl)ethanone;

1-((1-(methylsulfonyl)indolin-5-yl)methyl)-5,6,7,8-tetrahydro-1H-naphtho[2,3-d]imidazole;

(S)-5-((5,6,7,8-tetrahydro-1H-naphtho[2,3-d]imidazol-1-yl)methyl)-2,3-dihydro-1H-inden-2-ol;

(S)—N-methyl-5-((5,6,7,8-tetrahydro-1H-naphtho[2,3-d]imidazol-1-yl)methyl)-2,3-dihydro-1H-inden-2-amine;

(R)-1-(1-(1,2,3,4-tetrahydroisoquinolin-6-yl)ethyl)-5,6,7,8-tetrahydro-1H-naphtho[2,3-d]imidazole;

(R)-6-((R)-1-(5,6,7,8-tetrahydro-1H-naphtho[2,3-d]imidazol-1-yl)ethyl)chroman-3-ol;

(S)-6-amino-7-((R)-1-(5,6,7,8-tetrahydro-1H-naphtho[2,3-d]imidazol-1-yl)ethyl)-1,2,3,4-tetrahydronaphthalen-2-ol;

(S)-6-(methylamino)-7-((R)-1-(5,6,7,8-tetrahydro-1H-naphtho[2,3-d]imidazol-1-yl)ethyl)-1,2,3,4-tetrahydronaphthalen-2-ol; and

1-(4-methoxy-3-methylbenzyl)-1,5,6,7-tetrahydroindeno[5,6-d]imidazol-2-amine.

In another aspect, compounds of the invention have formula (II),

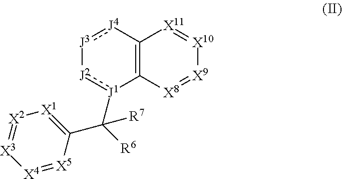

or a pharmaceutically acceptable salt, ester, amide, or prodrug form thereof,

wherein

J¹ is selected from C, CR^{51a}, and N;

J² is selected from CR^{52a}, C(R^{52b}R^{52c}), N, NR^{52d}, O, and S, or J² is absent;

J³ is selected from CR^{53a}, C(R^{53b}R^{53e}), N, NR^{53d}, O, and S, or J³ is absent;

J⁴ is selected from CR^{54a}, C(R^{54b}R^{54c}), N, NR^{54d}, O, and S, or J⁴ is absent;

- - - is an optional bond;

provided that valency of carbon, nitrogen, oxygen, and sulfur atoms are not exceeded;

provided that only one of J², J³, and J⁴ may be absent;

R^{51a}, R^{52a}, R^{52b}, R^{52c}, R^{52d}, R^{53a}, R^{53b}, R^{53c}, R^{53d}, R^{54a}, R^{54b}, R^{54c}, R^{54d} are each independently selected from the group consisting of —H, —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or

two adjacent J groups may together form a substituted or unsubstituted C₃-C₆ cycloalkyl, a substituted or unsubstituted heteroaryl, a substituted or unsubstituted heterocyclyl, or a substituted or unsubstituted aryl; and

X¹, X², X³, X⁴, X⁵, R⁶, R⁷, X⁸, X⁹, X¹⁰, and X¹¹ are as defined above.

In certain embodiments, X¹ is CR¹; X² is CR²; X³ is CR³; X⁴ is CR⁴; X⁵ is CR⁵; X⁸ is CR⁸; X⁹ is CR⁹; X¹⁰ is CR¹⁰; and X¹¹ is CR¹¹; wherein R¹, R⁴, R⁵, R⁶, R⁸, and R¹¹ are each hydrogen; wherein R⁷ is hydrogen or methyl; wherein R² is methyl; wherein R³ is methoxy; and wherein R⁹ and R¹⁰ together form an unsubstituted C₅-C₆-cycloalkyl.

In certain embodiments, the ring formed by J¹, J², J³, and J⁴, together with the carbon atoms to which J¹ and J⁴ are attached, is selected from:

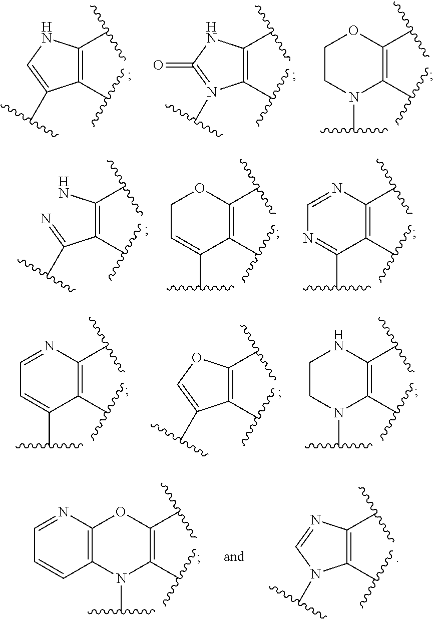

Further exemplary compounds of the invention include, but are not limited to,

3-(4-methoxy-3-methylbenzyl)-1,5,6,7-tetrahydrocyclopenta[f]indole;

1-(4-methoxy-3-methylbenzyl)-3,5,6,7-tetrahydroindeno[5,6-d]imidazol-2(1H)-one;

3-(4-methoxy-3-methylbenzyl)-1,5,6,7-tetrahydrocyclopenta[f]indazole;

4-(4-methoxy-3-methylbenzyl)-2,3,4,6,7,8-hexahydroindeno[5,6-b][1,4]oxazine;

4-(4-methoxy-3-methylbenzyl)-2,6,7,8-tetrahydrocyclopenta[g]chromene;

4-(4-methoxy-3-methylbenzyl)-7,8-dihydro-6H-cyclopenta[g]quinazoline;

4-(4-methoxy-3-methylbenzyl)-7,8-dihydro-6H-cyclopenta[g]quinoline;

3-(4-methoxy-3-methylbenzyl)-6,7-dihydro-5H-indeno[5,6-b]furan;

1-(4-methoxy-3-methylbenzyl)-2,3,4,6,7,8-hexahydro-1H-cyclopenta[g]quinoxaline; and

5-(4-methoxy-3-methylbenzyl)-5,7,8,9-tetrahydroindeno[5,6-b]pyrido[3,2-e][1,4]oxazine.

Compounds of the invention may contain asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the scope of this invention. The present invention is meant to comprehend all such isomeric forms of these compounds.

Compounds of the invention may be provided in a prodrug form. The term “prodrug” is art-recognized and is intended to encompass compounds which, under physiological conditions, are converted into the antibacterial agents of the present invention. A common method for making a prodrug is to select moieties which are hydrolyzed under physiological conditions to provide the desired compound. In other embodiments, the prodrug is converted by an enzymatic activity of the host animal or the target bacteria.

Compounds of the invention may be provided as a pharmaceutically acceptable salt. Pharmaceutically acceptable salts are salts that retain the desired biological activity of the parent compound and do not impart undesired toxicological effects. Examples of such salts are (a) acid addition salts organic and inorganic acids, for example, acid addition salts which may, for example, be hydrochloric acid, sulphuric acid, methanesulphonic acid, fumaric acid, maleic acid, succinic acid, acetic acid, benzoic acid, oxalic acid, citric acid, tartaric acid, carbonic acid, phosphoric acid, trifluoroacetic acid, formic acid and the like. Pharmaceutically acceptable salts can also be prepared from by treatment with inorganic bases, for example, sodium, potassium, ammonium, calcium, or ferric hydroxides, and such organic bases as isopropylamine, trimethylamine, 2-ethylamino ethanol, histidine, procaine, and the like. Pharmaceutically acceptable salts can also be formed from elemental anions such as chlorine, bromine and iodine.

Compounds of the invention which contain basic nitrogen-containing groups can be quaternized using agents such as (C₁-C₄)-alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, do-decyl, lauryl, myristyl and stearyl chloride, bromide and iodide; and aryl-(C₁-C₄)alkyl halides, for example benzyl chloride and phenethyl bromide. Both water- and oil-soluble compounds of the invention can be prepared using such salts.

The acid-addition salts of basic compounds of the invention can be prepared by bringing the free base form into contact with a sufficient amount of the desired acid, causing the formation of the salt in a conventional manner. The free base can be regenerated by bringing the salt form into contact with a base and isolating the free base in a conventional manner.

The base-addition salts of acidic compounds of the invention can be prepared by bringing the free acid form into contact with a sufficient amount of the desired base, causing the formation of the salt in a conventional manner. The free acid can be regenerated by bringing the salt form into contact with an acid and isolating the free acid in a conventional manner.

If a compound of the invention contains more than one group which is capable of forming pharmaceutically acceptable salts, the compounds of the invention also encompasses multiple salts. Typical multiple salt forms include, for example, bitartrate, diacetate, difumarate, dimeglumine, di-phosphate, disodium and trihydrochloride.

Compounds of the invention can be prepared in the form of their hydrates. The term “hydrate” includes but is not limited to hemihydrate, monohydrate, dihydrate, trihydrate, tetrahydrate and the like.

Compounds of the invention can be prepared in the form of a solvate with any organic or inorganic solvent, for example alcohols such as methanol, ethanol, propanol and isopropanol, ketones such as acetone, aromatic solvents and the like.

Compounds of the invention can be prepared in any solid or liquid physical form. For example, the compound can be in a crystalline form, in amorphous form, and have any particle size. Furthermore, the compound particles may be micronized, or may be agglomerated, particulate granules, powders, oils, oily suspensions or any other form of solid or liquid physical form.

Compounds of the invention may exhibit polymorphism. This invention further includes different polymorphs of the compounds of the present invention. The term “polymorph” refers to a particular crystalline state of a substance, having particular physical properties such as X-ray diffraction, IR spectra, melting point, and the like.

### Synthetic Methods

The compounds of the invention can be better understood in connection with the following synthetic schemes and methods which illustrate a means by which the compounds can be prepared.

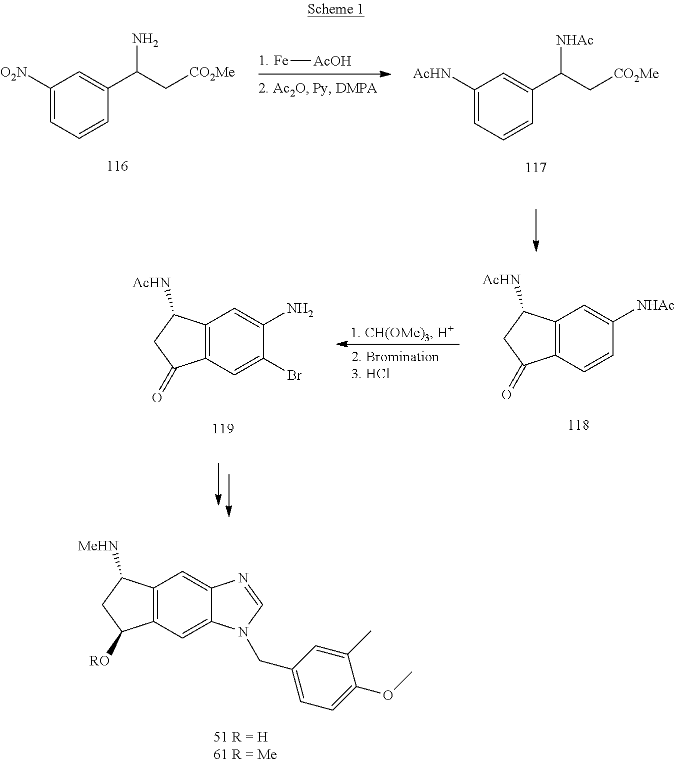

Compounds of formula 51 and 61 can be prepared as shown in Scheme 1 from the known amino ester 116. This aminoester can be transformed to bis-acetamide 117 by reduction of the nitro group using Fe in acetic acid, followed by acylation of the resulting amines with Ac₂O. Saponification of methyl ester 117 followed by Friedel-Craft's acylation will provide indanone derivative 118. Protection of the carbonyl group as a dimethyl ketal followed by bromination and deprotection of ketal will provide aminoketone 119. This can be converted to benzimidazole derivatives by following an established protocol. Further standard synthetic manipulations will provide proposed derivatives 51, 61, and other structural variants.

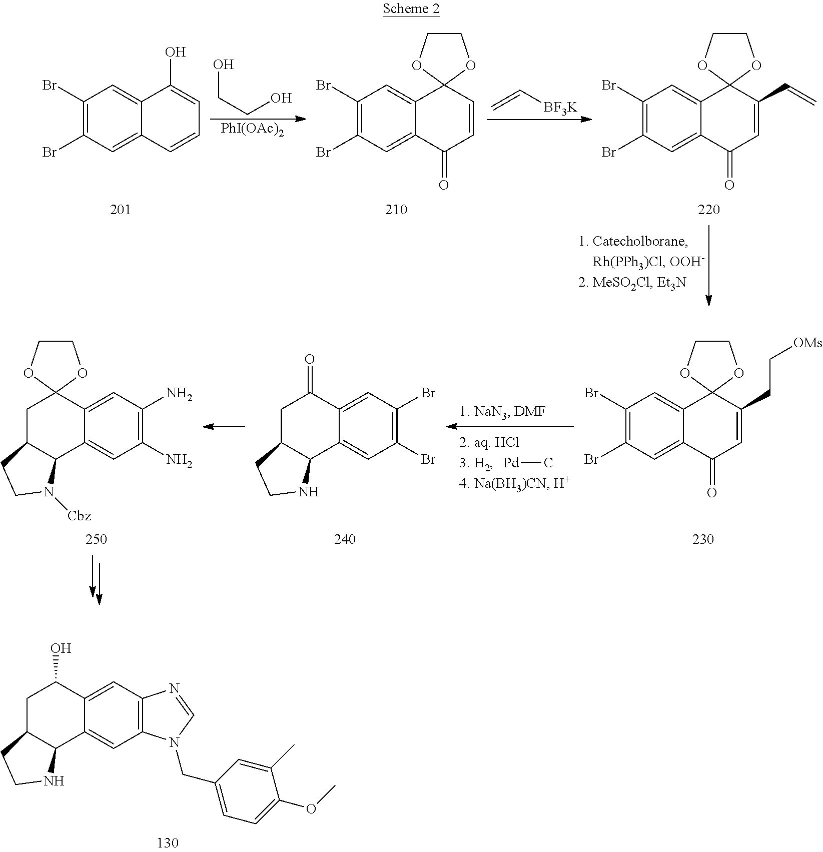

Compounds of formula 130 can be synthesized as shown in Scheme 2. Reaction of naphthol derivative 201 with ethylene glycol in the presence of PhI(OAc)₂ will provide enone 210. Treatment of 210 with potassium vinyltrifluoroborate using a protocol reported by Hayashi and co-workers would provide compound 220. Selective hydroboration of the terminal olefin using catecholborane in the presence of Rh(PPh₃)₃Cl, followed by mesylation of the resulting alcohol would provide mesylate 230. Displacement of the mesylate with sodium azide will provide the corresponding azide. Removal of the ketal with aqueous HCl followed by reduction of the azide and reductive amination will provide tetrahydrobenzo-indolone 240. Protection of the amine as a Cbz-derivative, and the carbonyl group as a ketal followed by treatment of the resulting dibromide with NH₄OH in the presence of a catalytic amount of Cu(I) as described by Jing and co-workers would result in bis-amine 250. Reaction of 250 with trimethylorthoformate can provide the corresponding benzimidazole derivative, which can be transformed into compound 130 using Mitsunobu reaction with the corresponding benzyl alcohol, deprotection of the ketal, and reduction of the resulting ketone.

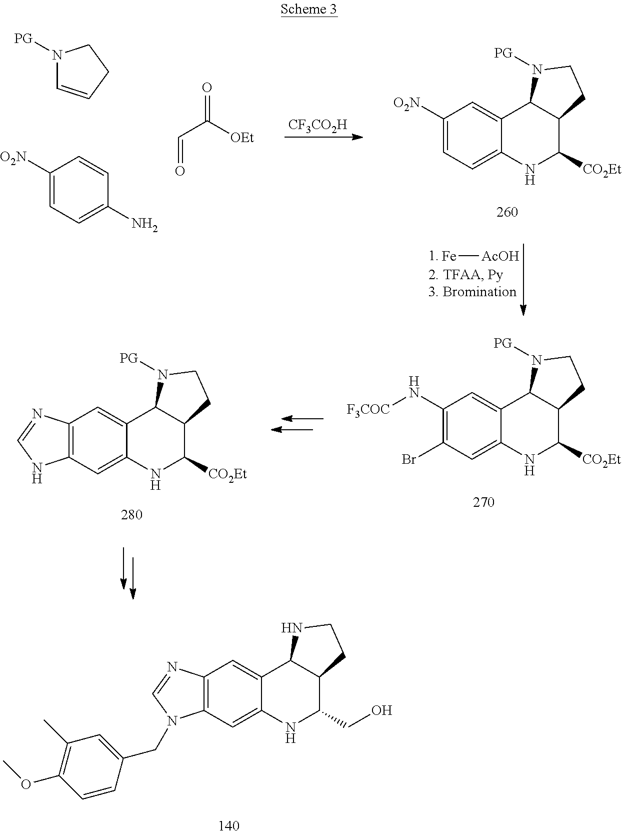

The synthesis compound 140 is outlined in Scheme 3. Tetrahydroquinoline derivative 260 can be prepared by a multicomponent reaction between p-nitroaniline, ethyl glyoxalate, and appropriately N-protected 2,3-dihydropyrrole in the presence of trifluoroacetic acid. Tetrahydroquinoline derivative 260 can be converted to bromoacetamide 270 by reduction of the nitro group, followed by acylation of the resulting amine with trifluoroacetic anhydride (TFAA), and subsequent bromination. Removal of the trifluoroacetamide group will provide the corresponding bromoamine which will be converted to benzimidazole derivative 280 using a known protocol. Benzimidazole derivative 280 can be converted to compound 140 by Mitsunobu reaction of 280 with an appropriate alcohol, removal of the amino protecting group, and reduction of the ester group.

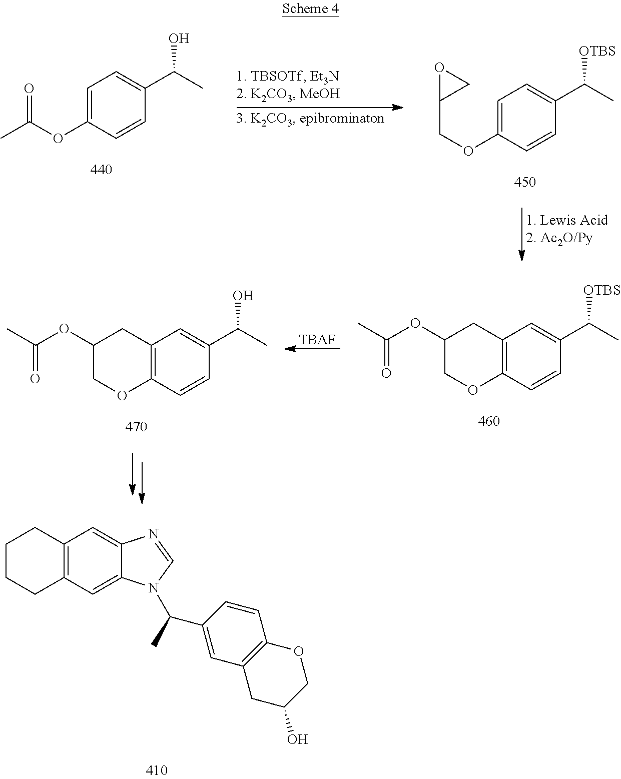

The synthesis of compound 410 is shown in Scheme 4. Treatment of the known alcohol 440 with TBSOTf in the presence of Et₃N will provide the corresponding TBS ether. Removal of the acetate group by treatment with K₂CO₃/MeOH followed by reaction of the resulting phenol derivative with epibromohydrin will provide epoxide 450. Lewis acid mediated tandem epoxide opening, Friedel-Crafts cyclization utilizing a protocol reported by Pericas and co-workers will provide the corresponding chroman derivative. Acylation of the resulting alcohol with Ac₂O will provide acetate 460. This can be transformed into alcohol 470 by removal of the silyl group with TBAF. This alcohol can be converted to proposed inhibitor 410 by Mitsunobu reaction with an appropriate benzimidazole derivative.

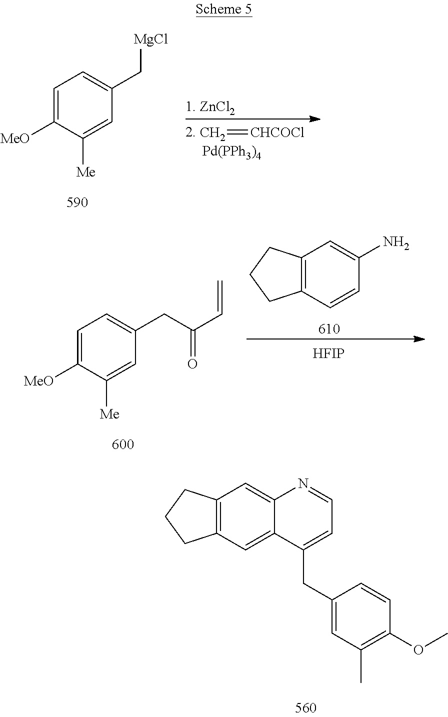

The synthesis of compound 560 is shown in Scheme 5. Treatment of magnesium chloride 590 with ZnCl₂ and subsequent reaction with acryloyl chloride in the presence of Pd(PPh₃)₄ can provide α,β-unsaturated ketone 600. Treatment of 600 with 5-aminoindan 610 in the presence of HFIP as described by Legros et. al., can afford compound 560.

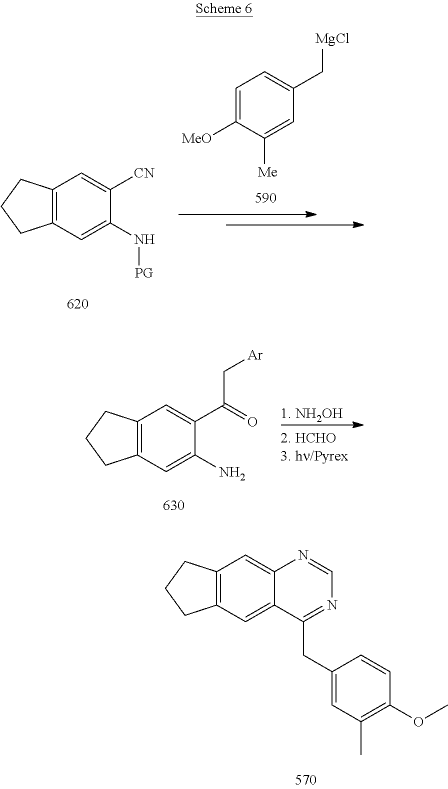

For the synthesis of inhibitor 570, known dihydroindene-5-carbonitrile 620 can be treated with 590 using a known protocol to provide ketone 630. Formation of the oxime, then reaction with HCHO, and subsequent irradiation can afford quinazoline derivative 570.

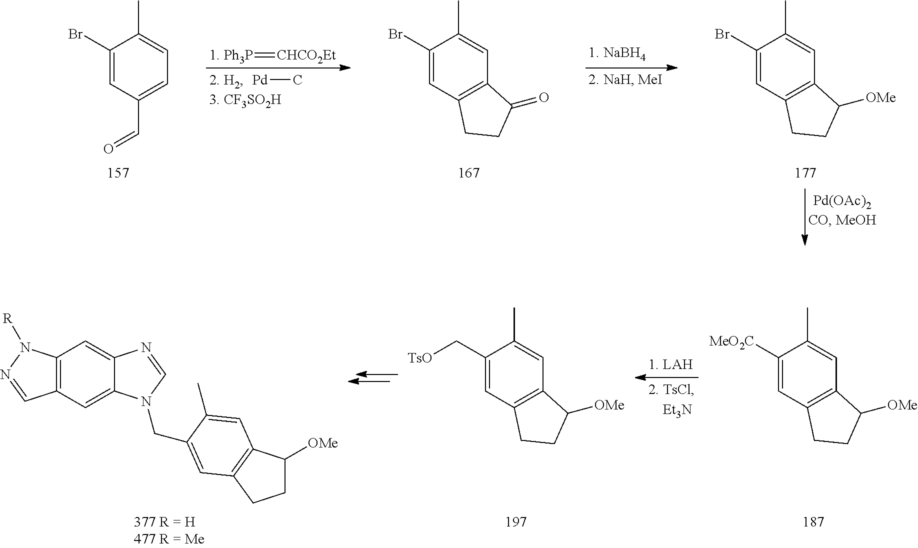

The indanylmethyl ligand synthesis for compounds 377 and 477 is shown in Scheme 7. The known 3-bromo-4-methylbenzaldehyde 157 will be converted to 2-indanone 167 by Wittig olefination, hydrogenation followed by Friedel-Craft acylation of the resulting acid. Reduction of the ketone followed by methylation of the resulting alcohol would provide derivative 177. Compound 177 can be transformed into methyl ester 187 by Pd mediated carboxymethylation. Reduction of the methyl ester followed by treatment of the resulting alcohol with TsCl would provide tosyl derivative 197. The corresponding benzimidazole derivative will be synthesized using established synthetic protocol. Alkylation of the benzimidazole with the tosyl derivative will furnish compounds 377 and 477.

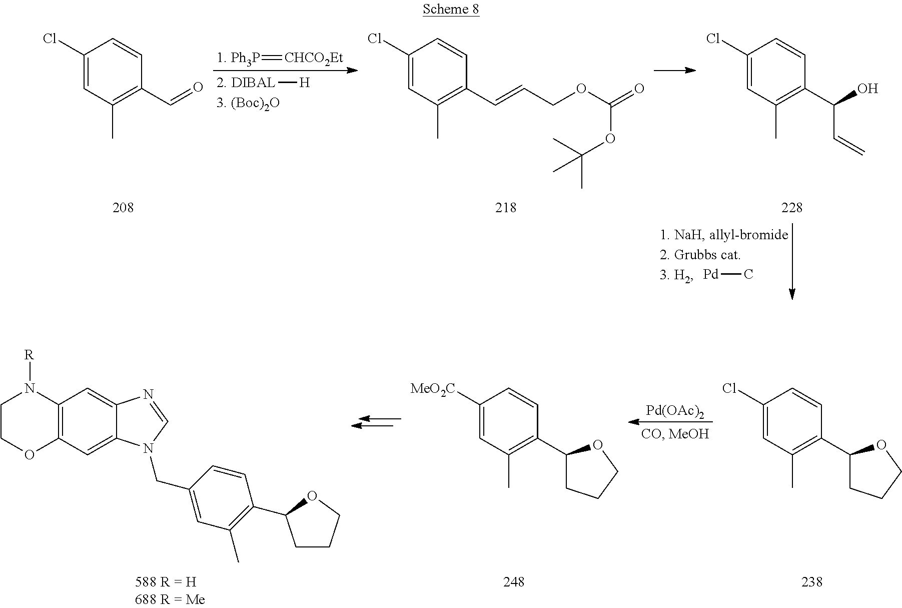

The synthesis of the corresponding tetrahydrofuranyl ligand of compounds 588 and 688 is shown in Scheme 8. Commercially available benzaldehyde 208 will be converted to allylic carbonate 218 by Wittig olefination, reduction, and treatment of the resulting alcohol with (Boc)₂O. Allylic carbonate 218 can be transformed into chiral allylic alcohol 228 using a protocol developed by Carreira and co-workers. Allylation of 228 with allyl bromide followed by Grubbs ring closing metathesis of the resulting ether followed by hydrogenation would provide tetrahydrofuran derivative 238. Compound 238 can be converted to compound 248 by Pd-mediated carboxymethylation. Ester 248 will be utilized in the synthesis of compounds 588 and 688 following the similar reaction sequence as shown for compound 377 in Scheme 7.

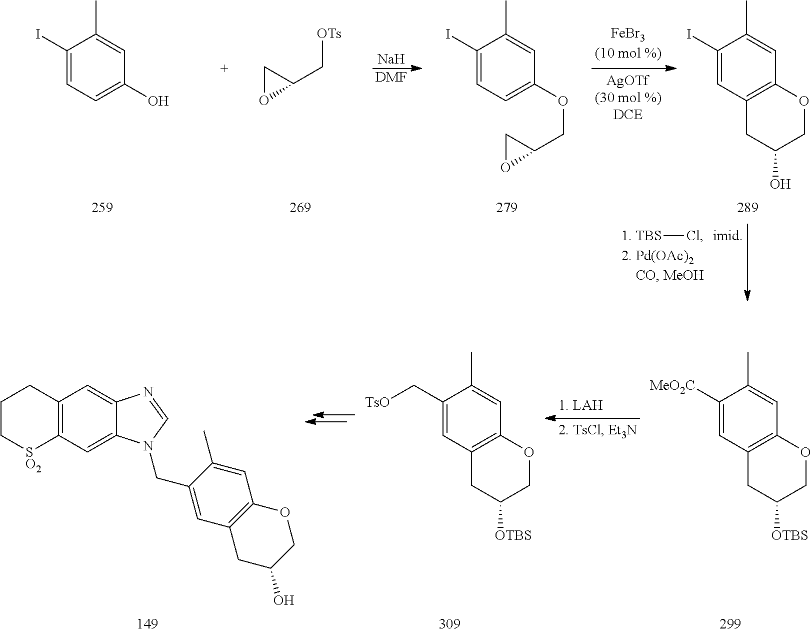

The synthesis of the corresponding tetrahydropyranyl ligand in compound 149 is shown in Scheme 9. Reaction of known 4-iodo-3-methylphenol 259 with epoxide 269 in the presence of NaH would provide corresponding arylglycidyl ether 279. Cyclization of compound 279 using a protocol developed by Pericas and co-workers would provide the key tetrahydropyran derivative 289. This compound can be transformed into methyl ester 299 by TBS protection followed by Pd-mediated carboxymethylation as described above. Reduction of the methyl ester followed by treatment of the resulting alcohol with TsCl would provide tosyl derivative 309. This will be utilized in the synthesis of compound 149 following the similar reaction sequence as shown for compound 377 in Scheme 7.

In certain embodiments, the products may be further modified, for example, by manipulation of substituents. These manipulations may include, but are not limited to, reduction, oxidation, organometallic cross-coupling, alkylation, acylation, and hydrolysis reactions which are commonly known to those skilled in the art. In some cases, the order of carrying out the foregoing reaction schemes may be varied to facilitate the reaction or to avoid unwanted reaction products.

### Pharmaceutical Compositions

Compounds of the invention may be provided in a pharmaceutical composition comprising a therapeutically effective amount of a compound of the invenion in combination with a pharmaceutically suitable carrier.

The antibacterial compositions of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compounds and compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. A drug delivery device for delivering aerosols may include a suitable aerosol canister with a metering valve containing a pharmaceutical aerosol formulation as described and an actuator housing adapted to hold the canister and allow for drug delivery. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise a compound of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In certain embodiments, compounds of the invention may be formulated as a tablet, pill capsule or other appropriate ingestible formulation (collectively hereinafter “tablet”), to provide a therapeutic dose in 10 tablets or fewer. In another example, a therapeutic dose is provided in 50, 40, 30, 20, 15, 10, 5 or 3 tablets.

In a certain embodiment, the antibacterial agent is formulated for oral administration as a tablet or an aqueous solution or suspension. In another embodiment of the tablet form of the antibacterial agent, the tablets are formulated such that the amount of antibacterial agent (or antibacterial agents) provided in 20 tablets, if taken together, would provide a dose of at least the median effective dose (ED50), e.g., the dose at which at least 50% of individuals exhibited the quantal effect of inhibition of bacterial cell growth or protection (e.g., a statistically significant reduction in infection). In a further embodiment, the tablets are formulated such that the total amount of antibacterial agent (or antibacterial agents) provided in 10, 5, 2 or 1 tablets would provide at least an ED50 dose to a patient (human or non-human mammal). In other embodiments, the amount of antibacterial agent (or antibacterial agents) provided in 20, 10, 5 or 2 tablets taken in a 24 hour time period would provide a dosage regimen providing, on average, a mean plasma level of the antibacterial agent(s) of at least the ED50 concentration (the concentration for 50% of maximal effect of, e.g., inhibiting bacterial cell growth). In other embodiments less than 100 times, 10 times, or 5 times the ED50 is provided. In other embodiments, a single dose of tablets (1-20 tablets) provides about 0.25 mg to 1250 mg of an antibacterial agent(s).

Likewise, the antibacterial agents can be formulated for parenteral administration, as for example, for subcutaneous, intramuscular or intravenous injection, e.g., the antibacterial agent can be provided in a sterile solution or suspension (collectively hereinafter “injectable solution”). The injectable solution is formulated such that the amount of antibacterial agent (or antibacterial agents) provided in a 200 cc bolus injection would provide a dose of at least the median effective dose, or less than 100 times the ED50, or less than 10 or 5 times the ED50. The injectable solution may be formulated such that the total amount of antibacterial agent (or antibacterial agents) provided in 100, 50, 25, 10, 5, 2.5, or 1 cc injections would provide an ED50 dose to a patient, or less than 100 times the ED50, or less than 10 or 5 times the ED50. In other embodiments, the amount of antibacterial agent (or antibacterial agents) provided in a total volume of 100 cc, 50, 25, 5 or 2 cc to be injected at least twice in a 24 hour time period would provide a dosage regimen providing, on average, a mean plasma level of the antibacterial agent(s) of at least the ED50 concentration, or less than 100 times the ED50, or less than 10 or 5 times the ED50. In other embodiments, a single dose injection provides about 0.25 mg to 1250 mg of antibacterial agent.

Compositions according to the invention can contain one or more additional agents, e.g., other antibiotics, anti-inflammatories, anti-fungals, steroids, decongestants, bronchodialators, and the like. For example, an anti-inflammatory drug or steroid can be co-administered such as ibuprofen, prednisone (corticosteroid) or pentoxifylline.

### Dosages and Dosing Schedules

The dosage regimen utilizing the compounds of the present invention can be selected in accordance with a variety of factors including type, species, age, weight, sex and the type of infection being treated; the severity (i.e., stage) of the disease to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the infection.

The amount of the compound administered to a patient is less than an amount that would cause unmanageable toxicity in the patient. In the certain embodiments, the amount of the compound that is administered to the patient is less than the amount that causes a concentration of the compound in the patient's plasma to equal or exceed the toxic level of the compound. In one embodiment, the concentration of the compound in the patient's plasma is maintained at about 10 nM. In another embodiment, the concentration of the compound in the patient's plasma is maintained at about 25 nM. In another embodiment, the concentration of the compound in the patient's plasma is maintained at about 50 nM. In another embodiment, the concentration of the compound in the patient's plasma is maintained at about 100 nM. In another embodiment, the concentration of the compound in the patient's plasma is maintained at about 500 nM. In another embodiment, the concentration of the compound in the patient's plasma is maintained at about 1000 nM. In another embodiment, the concentration of the compound in the patient's plasma is maintained at about 2500 nM. In another embodiment, the concentration of the compound in the patient's plasma is maintained at about 5000 nM. The optimal amount of the compound that should be administered to the patient in the practice of the present invention will depend on the particular compound used and the type of infection being treated.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

For oral administration, suitable daily dosages are for example between about 2-4000 mg administered orally once-daily, twice-daily or three times-daily, continuous (every day) or intermittently (e.g., 3-5 days a week). For example, when used to treat the desired disease, the dose of the compound can range between about 2 mg to about 2000 mg per day.

The compounds of the present invention may be administered once daily (QD), or divided into multiple daily doses such as twice daily (BID), and three times daily (TID). For administration once a day, a suitably prepared medicament would therefore contain all of the needed daily dose. For administration twice a day, a suitably prepared medicament would therefore contain half of the needed daily dose. For administration three times a day, a suitably prepared medicament would therefore contain one third of the needed daily dose.

In addition, the administration can be continuous, i.e., every day, or intermittently. The terms “intermittent” or “intermittently” as used herein means stopping and starting at either regular or irregular intervals.

Typically, an intravenous formulation may be prepared which contains a concentration of the compound of the invention of between about 1.0 mg/mL to about 10 mg/mL. In one example, a sufficient volume of intravenous formulation can be administered to a patient in a day such that the total dose for the day is between about 1 and about 1500 mg/mL.

Subcutaneous formulations, preferably prepared according to procedures well known in the art at a pH in the range between about 5 and about 12, also include suitable buffers and isotonicity agents. They can be formulated to deliver a daily dose of active compound in one or more daily subcutaneous administrations, e.g., one, two or three times each day.

The compounds can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

It should be apparent to a person skilled in the art that the various modes of administration, dosages and dosing schedules described herein merely set forth specific embodiments and should not be construed as limiting the broad scope of the invention. Any permutations, variations and combinations of the dosages and dosing schedules are included within the scope of the present invention.

### Efficacy of Treatment

The efficacy of treatment with the compounds and compositions may be determined in a number of fashions known to those of skill in the art.

In one exemplary method, the median survival rate of the bacteria or bacteria median survival time or life span for treatment with a subject composition may be compared to other forms of treatment with the particular FabI inhibitor, or with other antibiotic agents. The decrease in median bacteria survival rate or time or life span for treatment with a subject composition as compared to treatment with another method may be 10, 25, 50, 75, 100, 150, 200, 300, 400% even more. The period of time for observing any such decrease may be about 3, 5, 10, 15, 30, 60 or 90 or more days. The comparison may be made against treatment with the particular FabI inhibitor contained in the subject composition, or with other antibiotic agents, or administration of the same or different agents by a different method, or administration as part of a different drug delivery device than a subject composition. The comparison may be made against the same or a different effective dosage of the various agents. The different regiments compared may use measurements of bacterial levels to assess efficacy.

Alternatively, a comparison of the different treatment regimens described above may be based on the effectiveness of the treatment, using standard indices for bacterial infections known to those of skill in the art. One method of treatment may be 10%, 20%, 30%, 50%, 75%, 100%, 150%, 200%, 300% more effective, than another method.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using the same or different FabI inhibitor.

The antibacterial compositions of the present invention may inhibit bacterial FabI with a Ki of 5 μM or less, 1 μM or less, 100 nM or less, 10 nM or less or even 1 nM or less. In treatment of humans or other animals, the subject method may employ FabI inhibitors which are selective for the bacterial enzyme relative to the host animals' enoyl CoA hydratase, e.g., the Ki for inhibition of the bacterial enzyme is at least one order, two orders, three orders, or even four or more orders of magnitude less than the Ki for inhibition of enoyl CoA hydratase from the human (or other animal). That is, the practice of the subject method in vivo in animals utilizes FabI inhibitors with therapeutic indexes of at least 10, 100 or 1000.

Similarly, in the practice of the instant method, the antibacterial compounds of the present invention inhibit FabI with an IC₅₀ of 30 μM or less, 10 μM or less, 100 nM or less, or even 10 nM or less. In treatment of humans or other animals, the subject method may employ FabI inhibitors which are selective for the bacterial enzyme relative to the host animals' enoyl CoA hydratase, e.g., the IC₅₀ for inhibition of the bacterial enzyme is at least one order, two orders, three orders, or even four orders of magnitude less than the IC₅₀ for inhibition of enoyl CoA hydratase from the human (or other animal). That is, in preferred embodiments, the practice of the subject method in vivo in animals utilizes FabI inhibitors with therapeutic indexes of at least 10, 100 or 1000.

Alternatively, bacterial inhibition by an antibacterial compound of the present invention may also be characterized in terms of the minimum inhibitory concentration (MIC), which is the highest concentration of compound required to achieve complete inhibition of bacterial cell growth. Such values are well known to those in the art as representative of the effectiveness of a particular antibacterial agent against a particular organism or group of organisms. In the practice of the instant methods, the antibacterial compositions of the present invention inhibit bacterial growth with MIC values of 32 μg/mL or less, 16 μg/mL or less, 13 μg/mL or less, 10 μg/mL or less, 8 μg/mL or less, 2 μg/mL or less, 1 μg/mL or less, 0.5 μg/mL or less, 0.25 μg/mL or less, or 0.125 μg/mL or less. The value of MIC90, defined as the concentration of a compound required to inhibit the growth of 90% of bacterial strains within a given bacterial strain population, can also be used. In certain embodiments, the compounds of the present invention are selected for use based, inter alia, on having MIC90 values of less than about 32 μg/mL, less than about 16 μg/mL, less than about 8 μg/mL, less than about 4 μg/mL, less than about 2 μg/mL, less than about 1 μg/mL, less than about 0.5 μg/mL, less than about 0.25 μg/mL, or even less than about 0.125 μg/mL.

In other embodiments, the subject compounds are selected for use in animals, or animal cell/tissue culture based at least in part on having LD₅₀'s at least one order, or two orders, or three orders, or even four orders or more of magnitude greater than the ED₅₀. That is, in certain embodiments where the subject compounds are to be administered to an animal, a suitable therapeutic index is preferably greater than 10, 100, 1000 or even 10,000.

### Methods

The compounds and compositions of the invention can be used for treating or preventing bacterial infections. Compounds and compositions of the invention may be useful as antibacterial agents having a selective spectrum of activity in vitro and in vivo against bacterial strains relying on FabI and related targets. Such strains include Francisella tularensis, Staphylococcus aureus including multiresistant strains (such as methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains), Bacillus anthracis, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsia rickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia colt Haemophilus influenzae, Helicobacter pylori, Klebsiella pneumoniae, Neisseria meningitidis and also bacteria such as Mycobacterium tuberculosis carrying homologous FabI enzymes such as InhA or other organisms such as Plasmodium falciparum.

Compounds and compositions of the invention may exhibit antimicrobial activity via a dual mechanism of first depolarizing the bacterial cell membrane, then inhibiting FabI.

In certain embodiments, compounds of the invention may be used in the treatment of Staphylococcus aureus microbial infections including multiresistant strains such as methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains.

In certain embodiments, compounds of the invention may be used in the treatment of Francisella tularensis or Bacillus anthracis microbial infections.

In certain embodiments, compounds and compositions of the invention may be active against Gram positive strains, such as Enterococcus faecium, Staphylococcus epidermis, Staphylococcus saprophyticus, and Clostridium perfringens. In certain embodiments, compounds and compositions of the invention may be active against Gram negative strains, such as Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsia rickettsia, Salmonella enterica, Shigella sonnei, and Vibrio cholera. In certain embodiments, compounds and compositions of the invention may be active against non-stainable bacteria, such as Chlamydia trachomatis, Chlamydophila pneumonia, Chlamydophila psittaci, Mycobacterium tuberculosis, Mycobacterium leprae, and Mycobacterium ulcerans.

A method of treating or preventing a bacterial infection may include the step of administering to a subject in need thereof a therapeutically effective amount of a compound or composition of the invention. The subject may be a mammal The subject may be a human. The bacterial infection may be from a bacterium that expresses a FabI protein. The bacterial infection may be a human or animal infection caused by microbial pathogens selected from the group consisting of Francisella tularensis, Staphylococcus aureus including multiresistant strains (such as methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains), Bacillus anthracis, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsia rickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia colt Haemophilus influenzae, Helicobacter pylori, Klebsiella pneumoniae, Neisseria meningitidis and also bacteria such as Mycobacterium tuberculosis carrying homologous FabI enzymes such as InhA or other organisms such as Plasmodium falciparum, and any combination of the foregoing. In certain embodiments, the microbial pathogen is Staphylococcus aureus, such as methicillin-susceptible Staphylococcus aureus (MSSA), methicillin-resistant Staphylococcus aureus (MRSA), vancomycin-intermediate Staphylococcus aureus (VISA) or vancomycin-resistant Staphylococcus aureus (VRSA). In certain embodiments, the microbial pathogen is Francisella tularensis or Bacillus anthracis microbial infections.

Also disclosed herein is a method of treating or preventing tularemia, the method including administering to a subject in need thereof a therapeutically effective amount of a compound or composition of the invention.

Also disclosed herein is the use of a compound or composition of the invention for inhibiting Enoyl-ACP Reductase (FabI). The Enoyl-ACP Reductase (FabI) may be of Francisella tularensis, Staphylococcus aureus including multiresistant strains (such as methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains), Bacillus anthracis, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsia rickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia colt Haemophilus influenzae, Helicobacter pylori, Klebsiella pneumoniae, or Neisseria meningitidis.

As used herein, the term “therapeutically effective amount” means that amount of active compound or pharmaceutical agent that elicits the biological or medicinal response in a tissue, system, animal or human that is being sought by a researcher, veterinarian, medical doctor or other clinician. The therapeutic effect is dependent upon the disease or disorder being treated or the biological effect desired. As such, the therapeutic effect can be a decrease in the severity of symptoms associated with the disease or disorder and/or inhibition (partial or complete) of progression of the disease. The amount needed to elicit the therapeutic response can be determined based on the age, health, size and sex of the subject. Optimal amounts can also be determined based on monitoring of the subject's response to treatment. In addition, a therapeutically effective amount can be an amount that inhibits Enoyl-ACP Reductase (FabI).

Also disclosed herein is a method of manufacturing a medicament for the treatment of microbial infections using a compound of the invention.

Also disclosed herein is a method of disinfecting an inanimate surface comprising administering to the inanimate surface a compound or composition of the invention.

### Kits

This invention also provides kits for conveniently and effectively implementing the methods of this invention. Such kits may include a compound or composition of the invention, and optionally one or more of instructions, packaging, and dispensers. Kit components may be packaged for either manual or partially or wholly automated practice of the foregoing methods. In other embodiments involving kits, this invention contemplates a kit including compositions of the present invention, and optionally instructions for their use.

The compounds, compositions, methods and processes of the invention will be better understood by reference to the following examples, which are intended as an illustration of and not a limitation upon the scope of the invention.

### Biological Activity

Compounds of the invention were synthesized using methods disclosed herein and by methods known in the art for preparing benzimidazoles, for example. Purity of the compounds was assessed by LC-MS and/or NMR to be >95%. Stock solutions (50 mM) of each compound were prepared in dimethyl sulfoxide (DMSO) and diluted to the required working concentrations. The substrate for FabI, butenyl Co-A, the co-factor NADH, and DMSO were purchased from Sigma-Aldrich (St. Louis, Mo.).

Compounds of the present invention are modulators of the NADH-dependent enzyme, enoyl-ACP reductase I (FabI). When the FabI enzyme is inhibited by the compounds of the present invention, FabI is unable to exert its enzymatic, biological and/or pharmacological effects. The compounds of the present invention are therefore useful in the treatment and prevention of bacterial infections, and in particular, infections related to organisms that rely on FabI. Compounds of the invention exhibit an inhibition (IC₅₀) with regard to FabI of 10 μM or less, 5 μM or less, 1 μM or less, 0.5 μM or less, 0.3 μM or less, 0.1 μM or less, 0.05 μM or less, 0.01 μM or less, or 0.005 μM or less. Compounds of the invention may have Kᵢ values 1 μM or less, 1 μM or less, or 0.1 μM or less. Kᵢ values may be calculated from the IC₅₀s using the Cheng-Prusoff equation.

### Protein Expression and Purification

The gene for FabI (from F. tularensis subsp. tularensis Schu4) was commercially synthesized (Bio Basic Inc., Canada) after codon optimization. The gene was ligated into a pET-I5b vector with an N-terminal His tag and transformed into Escherichia coli BL21 (DE3) cells. The cells were grown at 310 K and induced with 1 mM IPTG when the OD reached 0.5. The cells were harvested after an additional 4 h of growth. Sonication was used to lyse the cells and the supernatant was loaded onto a nickel-chelated His-Trap column (GE Healthcare) and eluted with a stepwise gradient of imidazole in 50 mM Tris, 500 mM NaCl pH 8.0. The final purification step used a size-exclusion column (Superdex-200 26/60 from GE Healthcare) previously equilibrated with buffer consisting of 50 mM Tris, 100 mM NaCl pH 8.0 with 1 mM DTT. The FabI enzyme was soluble when over-expressed, and was purified to >98% purity.

### Enzymatic Assay

The FabI reaction converts one molecule of NADH and crotonyl-CoA into NAD⁺ and butyryl-CoA. The assay was conducted at room temperature in a 384-well plate. 40 μL of the assay solution contains 50 mM Tris pH 8, 100 mM NaCl, 0.1 mg/mL BSA, 0.01% triton, 2 mM TCEP, 200 μM NADH, 200 μM crotonyl-CoA and 0.2 μM of purified FtuFabI. The reaction was initiated by the addition of 10 μL of crotonyl-CoA. Enzyme activity was recorded using a BMG LabTech plate reader and following the rate of decrease in fluorescence of NADH at 450 nm (excitation wavelength 340 nm). The IC₅₀ values for compounds were determined by varying the concentrations of inhibitor in the assay solution. It was determined that <3% DMSO in the assay solution does not affect enzyme activity; hence 1 μL of the inhibitor dissolved in 100% DMSO was added to the assay solution. Reactions were allowed to pre-incubate in the assay solution for 30 minutes before adding 10 μL of crotonyl-CoA (final concentration of 200 μM) to initiate the reaction. The resulting data were used to calculate the percent inhibition values versus the non-inhibited control which consisted of DMSO without compound. The percent inhibition (% I) was determined using the equation % I=((A_{C}−A_{I})/A_{C})*100 where A_{C}=activity of the control (uninhibited) and A_{I}=activity with the inhibitor. The percent inhibition data were plotted as a function of inhibitor concentration and the data were fit via non-linear regression to the Hill equation using the Origin software.

Table 1 shows IC₅₀ values of compounds of the invention.

TABLE 1
Inhibition of FabI

| Compound ID | Structure | IC₅₀ (μM) |
|---|---|---|
| Compound 1 | 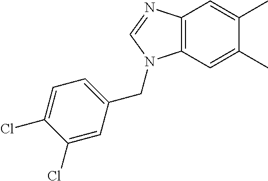 [SMILES: Cc1cc2ncn(Cc3ccc(Cl)c(Cl)c3)c2cc1C] | 0.3 |
| Compound 2 | 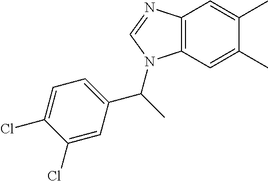 [SMILES: Cc1cc2ncn(C(C)c3ccc(Cl)c(Cl)c3)c2cc1C] | 0.37 |
| Compound 3 | 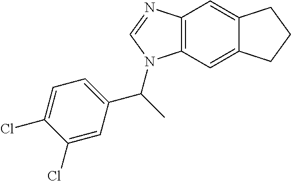 [SMILES: CC(c1ccc(Cl)c(Cl)c1)n1cnc2cc3c(cc21)CCC3] | 0.043 |
| Compound 4 | 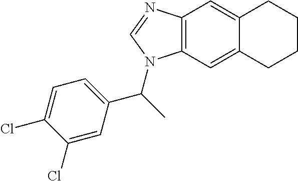 [SMILES: CC(c1ccc(Cl)c(Cl)c1)n1cnc2cc3c(cc21)CCCC3] | 0.63 |
| Compound 5 | 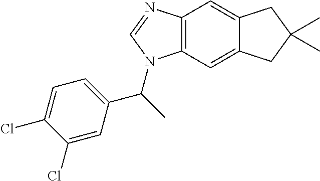 [SMILES: CC(c1ccc(Cl)c(Cl)c1)n1cnc2cc3c(cc21)CC(C)(C)C3] | 0.24 |
| Compound 6 | 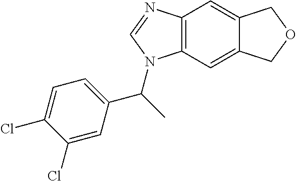 [SMILES: CC(c1ccc(Cl)c(Cl)c1)n1cnc2cc3c(cc21)COC3] | 0.89 |
| Compound 7 | 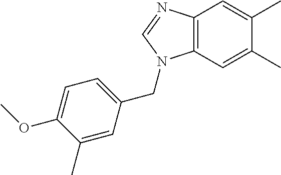 | 0.25 |
| Compound 8 | 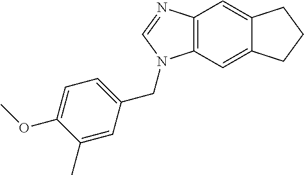 [SMILES: COc1ccc(Cn2cnc3cc4c(cc32)CCC4)cc1C] | 0.005 |
| Compound 9 | 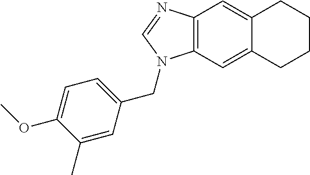 [SMILES: COc1ccc(Cn2cnc3cc4c(cc32)CCCC4)cc1C] | 0.14 |
| Compound 10 | 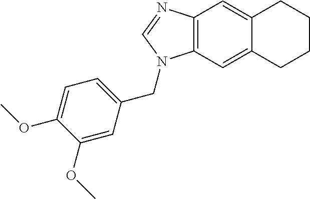 [SMILES: COc1ccc(Cn2cnc3cc4c(cc32)CCCC4)cc1OC] | 1.36 |
| Compound 11 | 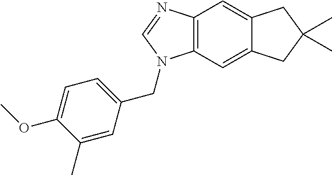 | 0.014 |
| Compound 12 | 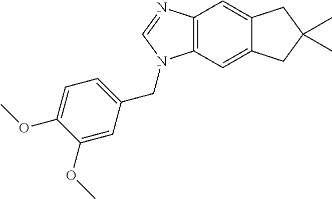 | 0.071 |
| Compound 13 | 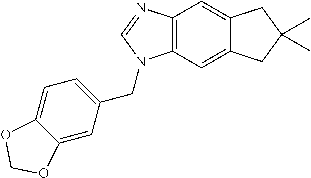 [SMILES: CC1(C)Cc2cc3ncn(Cc4ccc5c(c4)OCO5)c3cc2C1] | 0.029 |
| Compound 14 | 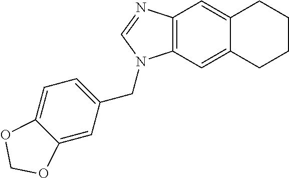 [SMILES: c1cc2c(cc1Cn1cnc3cc4c(cc31)CCCC4)OCO2] | 0.32 |
| Compound 15 | 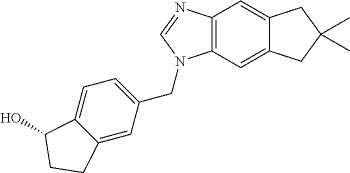 [SMILES: CC1(C)Cc2cc3ncn(Cc4ccc5c(c4)CC[C@@H]5O)c3cc2C1] | 0.041 |
| Compound 16 | 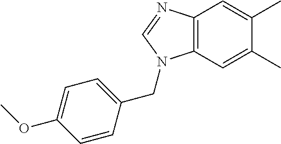 | 4.7 |
| Compound 17 | 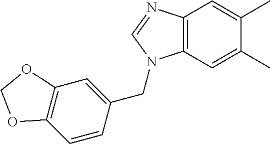 [SMILES: Cc1cc2ncn(Cc3ccc4c(c3)OCO4)c2cc1C] | 4.4 |
| Compound 18 | 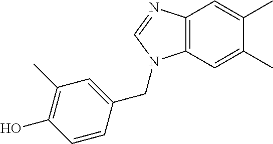 [SMILES: Cc1cc2ncn(Cc3ccc(O)c(C)c3)c2cc1C] | 1.3 |
| Compound 19 | 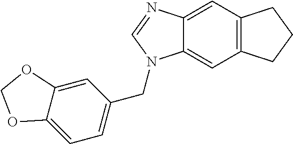 [SMILES: c1cc2c(cc1Cn1cnc3cc4c(cc31)CCC4)OCO2] | 2.2 |
| Compound 20 | 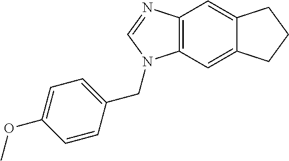 [SMILES: COc1ccc(Cn2cnc3cc4c(cc32)CCC4)cc1] | 0.84 |
| Compound 21 | 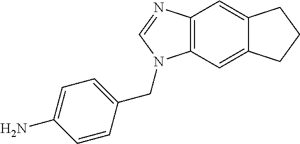 [SMILES: Nc1ccc(Cn2cnc3cc4c(cc32)CCC4)cc1] | 12 |
| Compound 22 | 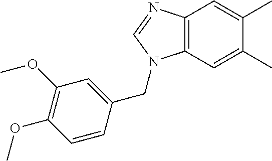 | 7.6 |
| Compound 23 | 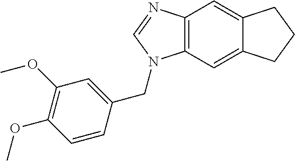 [SMILES: COc1ccc(Cn2cnc3cc4c(cc32)CCC4)cc1OC] | 5.9 |
| Compound 24 | 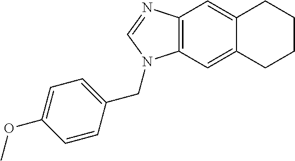 [SMILES: COc1ccc(Cn2cnc3cc4c(cc32)CCCC4)cc1] | 0.33 |

Compounds of the invention display competitive inhibition with respect to the substrate and uncompetitive inhibition with respect to the cofactor NADH. Ki values were determined for several compounds of the invention and the results are shown in Table 2. Ki values were calculated through rigorous experiments where concentrations of the substrate butenyl-CoA, and compounds, were varied as a function of each other at a saturating concentration of 200 μM of NADH. Experimental Ki values were found to be very close to the IC₅₀ values. Calculating Ki values using the Cheng-Prusoff equation provided similar Ki values. In the case of competitive inhibitors, the IC₅₀ values approximate Ki when the substrate concentration used is much lower than the Km.

TABLE 2
Ki Values

| Compound | Kᵢ¹(μM) | IC₅₀(μM) | Kᵢ ²(μM) |
|---|---|---|---|
| 4 | 0.57 ± 0.14 | 0.63 | 0.51 |
| 9 | 0.13 ± 0.01 | 0.14 | 0.11 |
| 10 | 1.43 ± 0.27 | 1.36 | 1.10 |
| 14 | 0.33 ± 0.04 | 0.32 | 0.26 |

¹Experimental Ki;
² Calculated using the Cheng-Prusoff Equation: IC₅₀ = Kᵢ (1 + [S]/Kₘ), where [S] = 200 μM and Kₘ = 830 μM.

### Antibacterial Assay

Compounds of the invention are useful antibacterial agents having a selective spectrum of activity in vitro against bacterial strains relying on FabI and related targets. Compounds of the invention show activity against F. tularensis, B. anthracis, and S. aureus including multiresistant strains.

The minimum inhibitory concentrations (MIC) were determined using the CLSI microdilution method against the following organisms: F. tularensis, strain Utah 112; B. anthracis, ΔANR strain; S. aureus (ATCC number 29213); MRSA (ATCC number 43300); E. coli (BW251113 strain) and E. coli TolC-, an efflux defective mutant. Compounds were serially diluted in LB medium across the rows of a 96-well plate. The appropriate bacterial cultures were grown to mid log-phase and then diluted to an OD₆₀₀=0.004 with fresh LB medium. These cultures were then added to each well, and the plate was incubated at 37° C. overnight without shaking. For each compound, the minimum concentration at which the first well had no signs of visible growth was reported as the MIC value. For each compound with an MIC<12.5 ug/mL, the minimum bactericidal concentration (MBC) was determined by plating all clear wells from the MIC assay onto LB agar, medium and identifying the lowest concentration at which no viable bacteria were detected by colony formation (the MBC).

Table 3 shows antibacterial activity of compounds of the invention against several organisms. E. coli TolC- is the E. coli efflux pump knockout mutant. MIC is the minimum inhibitory concentration while MBC is the minimum bactericidal concentration. MBCs were not determined for those compounds that displayed MICs≧12.5 μg/mL.

TABLE 3
Antibacterial Activity

| | MIC/MBC (both in μg/mL) | | | | | |
|---|---|---|---|---|---|---|
| Compound | F. tularensis | B. anthracis | S. aureus | MRSA | E. coli | E. coli TolC- |
| 2 | >12.5 | 12.5 | 12.5 | 12.5 | >12.5 | 6.3/12.5 |
| 3 | 3.9/9.4 | 3.9/4.7 | 5.5/>25.0 | 6.3/12.5 | >12.5 | 3.1/12.5 |
| 4 | 3.1/25.0 | 3.1/3.1 | 3.1/25.0 | 3.1/25.0 | >12.5 | 3.1/>25.0 |
| 5 | >12.5 | >12.5 | 4.7/>12.5 | 4.7/>12.5 | >12.5 | >12.5 |
| 6 | >12.5 | >12.5 | >12.5 | >12.5 | >12.5 | >12.5 |
| 7 | >12.5 | >12.5 | >12.5 | >12.5 | >12.5 | 12.5 |
| 8 | 5.5/12.5 | >12.5 | >12.5 | >12.5 | >12.5 | 12.5 |
| 9 | 3.1/6.3 | 6.3/6.3 | 6.3/25.0 | 12.5 | >12.5 | 4.7/9.4 |
| 10 | >12.5 | >12.5 | >12.5 | >12.5 | >12.5 | >12.5 |
| 11 | >12.5 | >12.5 | >12.5 | >12.5 | >12.5 | >12.5 |
| 12 | >12.5 | >12.5 | >12.5 | >12.5 | >12.5 | >12.5 |
| 13 | >12.5 | >12.5 | >12.5 | >12.5 | >12.5 | 12.5 |
| 14 | 4.7/18.8 | >12.5 | 4.7/>25.0 | 6.3/>25.0 | >12.5 | 9.4/25.0 |
| 15 | >12.5 | >12.5 | >12.5 | >12.5 | >12.5 | >12.5 |
| 19 | 6.3/37.5 | >12.5 | 9.4/>25.0 | 10.9/>25.0 | >12.5 | >12.5 |
| 20 | 5.5/37.5 | >12.5 | >12.5 | >12.5 | >12.5 | >12.5 |
| 21 | >12.5 | >12.5 | >12.5 | >12.5 | >12.5 | >12.5 |
| 24 | 4.7/18.8 | >12.5 | >12.5 | >12.5 | >12.5 | 12.5/>25.0 |

### Membrane Depolarization

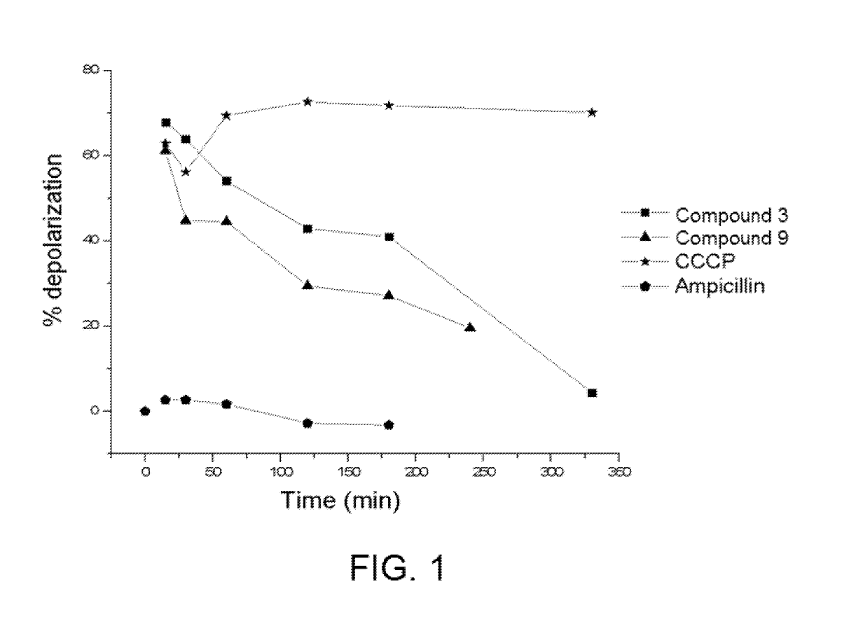

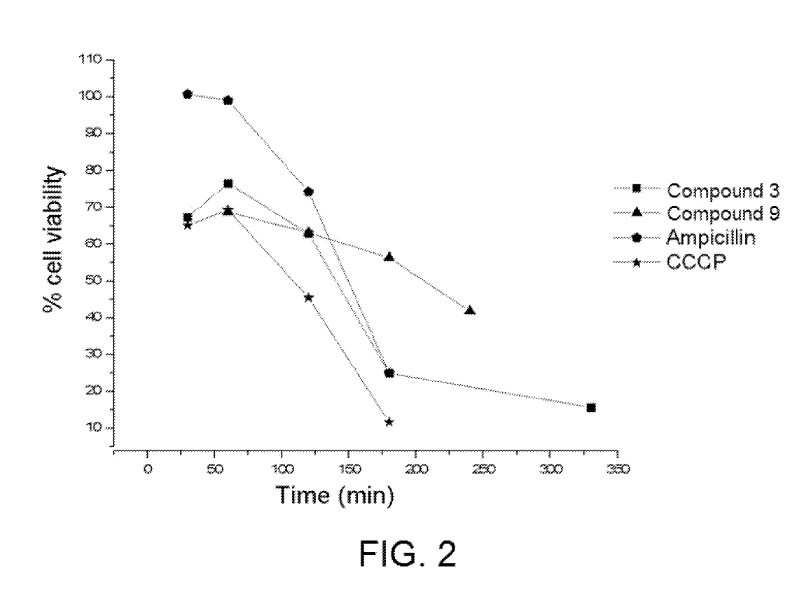

Membrane depolarization and cell viability were simultaneously measured as a function of time and it was found that compounds of the invention rapidly induce substantial depolarization of the S. aureus membrane within the first 30 min of exposure, with depolarization then more slowly decreasing over time, as shown in FIG. 1. This rapid initial depolarization is comparable to that of the well-known membrane depolarizer, carbonyl cyanide m-chlorophenylhydrazone (CCCP), a proton ionophore that reduces membrane potential to zero. The cell viability is comparable to that of CCCP, initially at 60-70% viability, but less than ampicillin, as shown in FIG. 2. This data suggest that compounds of the invention, which have a strong lipophilic character, insert into the membrane causing depolarization.

TABLE 4
Membrane Depolarization

| | | MIC | Depolarization | Cell viability |
|---|---|---|---|---|
| Name | Structure | (μg/mL) | (done at 2X MIC) | (done at 2X MIC) |
| Ampicillin | | 0.4 | No depolarization | bactericidal |
| | | | seen | |
| CCCP | | 1 | Highly depolarizing | bactericidal |
| Compound 3 | 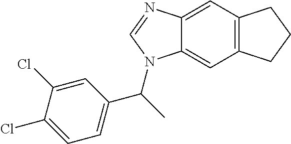 [SMILES: CC(c1ccc(Cl)c(Cl)c1)n1cnc2cc3c(cc21)CCC3] | 5.5 | Initially depolarizing (in first 60 min) but cells ‘recover’ and regain polarization compared to control with no compound | Bacteriostatic |
| Compound 9 | 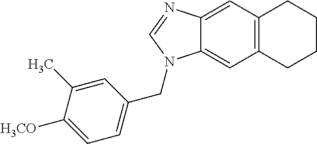 [SMILES: COc1ccc(Cn2cnc3cc4c(cc32)CCCC4)cc1C] | 6.3 | Initially depolarizing (in first 60 min) but cells ‘recover’ and regain polarization compared to control with no compound | Clearly bacteriostatic |
| Compound 4 | 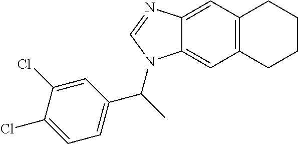 [SMILES: CC(c1ccc(Cl)c(Cl)c1)n1cnc2cc3c(cc21)CCCC3] | 3.1 | Initially depolarizing (in first 60 min) but cells ‘recover’ and regain polarization compared to control with no compound | Clearly bacteriostatic |
| Compound 14 | 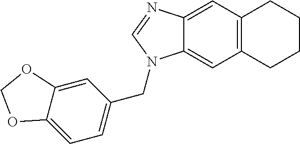 [SMILES: c1cc2c(cc1Cn1cnc3cc4c(cc31)CCCC4)OCO2] | 4.7 | Clearly no depolarization seen | Clearly bacteriostatic |
| Compound 1 | 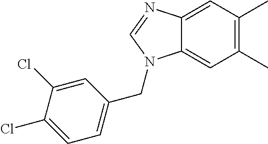 [SMILES: Cc1cc2ncn(Cc3ccc(Cl)c(Cl)c3)c2cc1C] | 25 | Depolarization seen initially in the first 60 min | Bactericidal intially but cells recover and compound is not lethal overnight |
| Triclosan | 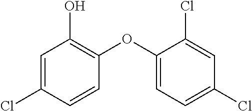 [SMILES: Oc1cc(Cl)ccc1Oc1ccc(Cl)cc1Cl] | 0.4 | Strongly depolarizing | Possibly bacteriostatic initially but then bactericidal after overnight incubation |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A compound of formula (I), 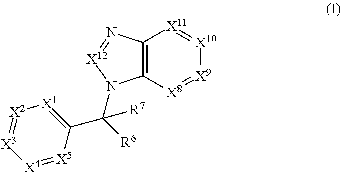
or a pharmaceutically acceptable salt, ester, amide, or prodrug form thereof,
wherein
X¹ is selected from CR¹ and N; X² is selected from CR² and N; X³ is selected from CR³ and N; X⁴ is selected from CR⁴ and N; X⁵ is selected from CR⁵ and N; X⁸ is selected from CR⁸ and N; X⁹ is selected from CR⁹ and N; X¹⁰ is selected from CR¹⁰ and N; X¹¹ is selected from CR11 and N; X¹² is selected from CR¹² and N;
R¹, R², R³, R⁴, and R⁵ are each independently selected from the group consisting of —H, —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or
R¹ and R², R² and R³, R³ and R⁴, or R⁴ and R⁵ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl;
R⁶ and R⁷ are each independently selected from the group consisting of —H, halo, and C₁-C₆-alkyl;
R⁸, R⁹, R¹⁰, and R¹¹ are each independently selected from the group consisting of —H, —OH, —NH₂, —NO₂, —CN, halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆alkoxy, halo-C₁-C₆alkoxy, hydroxy-C₁-C₆-alkoxy, C₁-C₆-alkoxy-C₁-C₆-alkoxy, C₁-C₆-alkylcarbonyl, C₁-C₆alkoxycarbonyl, C₁-C₆alkylcarbonyloxy, C₁-C₆alkyl-NHSO₂—, C₁-C₆alkyl-SO₂NH—, C₁-C₆alkyl-SO₂—, C₁-C₆alkylamino, di(C₁-C₆alkyl)amino, C₂-C₆alkenyl, C₂-C₆alkynyl, substituted or unsubstituted C₃-C₆ cycloalkyl, substituted or unsubstituted heteroaryl, substituted or unsubstituted heterocyclyl, and substituted or unsubstituted aryl, or
R⁸ and R⁹, R⁹ and R¹⁰, or R¹⁰ and R¹¹ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl; and
R¹² is selected from the group consisting of —H, —OH, —NH₂, halo, and C₁-C₆alkyl.

2. The compound of claim 1, wherein
X¹ is CR¹;
X² is CR²;
X³ is CR³;
X⁴ is CR⁴;
X⁵ is CR⁵;
X⁸ is CR⁸;
X⁹ is CR⁹;
X¹⁰ is CR¹⁰;
X¹¹ is CR¹¹; and
X¹² is CR¹².

3. The compound of claim 1, wherein
R⁸ is —H; and
R11 is —H.

4. The compound of claim 1, wherein
R¹ is —H; and
R⁵ is —H.

5. The compound of claim 1, wherein
R¹² is —H.

6. The compound of claim 1, wherein
R⁶ is —H; and
R⁷ is —H.

7. The compound of claim 1, wherein
R⁶ is —H; and
R⁷ is methyl.

8. The compound of claim 1, wherein
R⁴ is hydrogen.

9. The compound of claim 1, wherein
R² and R³ are each independently selected from the group consisting of halo, C₁-C₆-alkyl, halo-C₁-C₆-alkyl, hydroxy-C₁-C₆-alkyl, amino-C₁-C₆-alkyl, C₁-C₆-alkylamino-C₁-C₆-alkyl, di(C₁-C₆-alkyl)amino-C₁-C₆-alkyl, C₁-C₆-alkoxy-C₁-C₆-alkyl, C₁-C₆-alkoxy, halo-C₁-C₆-alkoxy, hydroxy-C₁-C₆-alkoxy, C₁-C₆-alkoxy-C₁-C₆-alkoxy, C₁-C₆-alkylcarbonyl, C₁-C₆-alkoxycarbonyl, C₁-C₆-alkylcarbonyloxy, C₁-C₆-alkyl-S—, C₁-C₆-alkyl-NHSO₂—, C₁-C₆-alkyl-SO₂NH—, C₁-C₆-alkyl-SO₂—, C₁-C₆-alkylamino, di(C₁-C₆-alkyl)amino, C₂-C₆-alkenyl, C₂-C₆-alkynyl, substituted or unsubstituted C₃-C₆ cycloalkyl, substituted or unsubstituted heteroaryl, substituted or unsubstituted heterocyclyl, and substituted or unsubstituted aryl, or
R² and R³ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl.

10. The compound of claim 1, wherein
R² and R³ are each independently selected from the group consisting of halo, C₁-C₆-alkyl, and C₁-C₆-alkoxy, or
R² and R³ together with the carbon atoms to which they are attached form a substituted or unsubstituted C₃-C₆ cycloalkyl, or a substituted or unsubstituted heterocyclyl.

11. The compound of claim 1, wherein
R² is methyl; and
R³ is methoxy.

12. The compound of claim 1, wherein
R² and R³ together with the carbon atoms to which they are attached form an unsubstituted methylenedioxy ring.

13. The compound of claim 1, wherein
R² is chloro; and
R³ is chloro.

14. The compound of claim 1, wherein
R⁹ and R¹⁰ are each independently selected from the group consisting of C₁-C₆-alkyl, or
R⁹ and R¹⁰ together with the carbon atoms to which they are attached form a substituted or unsubstituted C₃-C₆ cycloalkyl, a substituted or unsubstituted heteroaryl, a substituted or unsubstituted heterocyclyl, or a substituted or unsubstituted aryl.

15. The compound of claim 1, wherein
R⁹ is methyl; and
R¹⁰ is methyl.

16. The compound of claim 1, wherein
R⁹ and R¹⁰ together with the carbon atoms to which they are attached form a substituted or unsubstituted C₃-C₆ cycloalkyl.

17. The compound of claim 1, wherein
R⁹ and R¹⁰ together with the carbon atoms to which they are attached form an unsubstituted cyclopentyl ring.

18. The compound of claim 1, wherein
R⁹ and R¹⁰ together with the carbon atoms to which they are attached form an unsubstituted cyclohexyl ring.

19. The compound of claim 1, selected from the group consisting of: 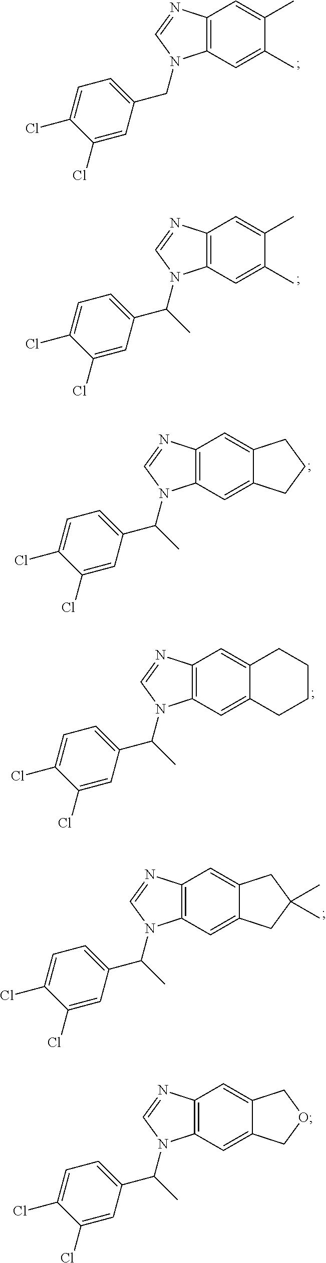 [SMILES: Cc1cc2ncn(Cc3ccc(Cl)c(Cl)c3)c2cc1C ; Cc1cc2ncn(C(C)c3ccc(Cl)c(Cl)c3)c2cc1C ; CC(c1ccc(Cl)c(Cl)c1)n1cnc2cc3c(cc21)CCC3 ; CC(c1ccc(Cl)c(Cl)c1)n1cnc2cc3c(cc21)CCCC3 ; CC(c1ccc(Cl)c(Cl)c1)n1cnc2cc3c(cc21)CC(C)(C)C3 ; CC(c1ccc(Cl)c(Cl)c1)n1cnc2cc3c(cc21)COC3] 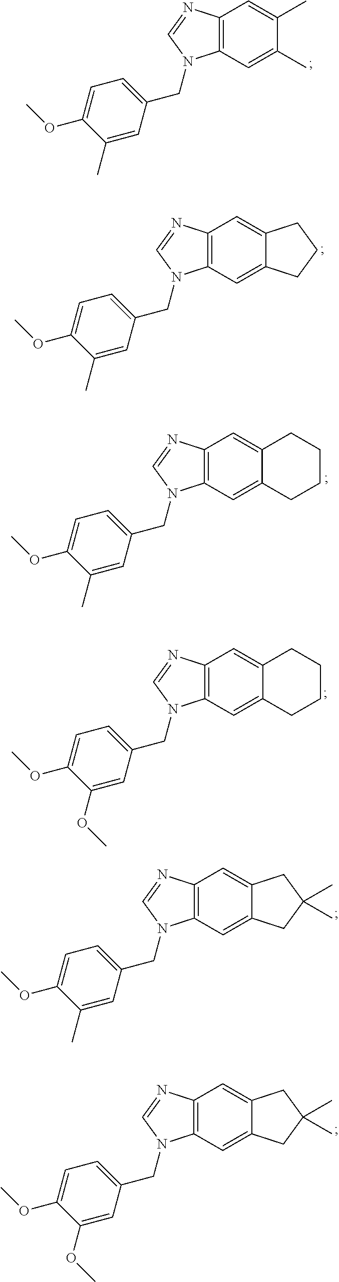 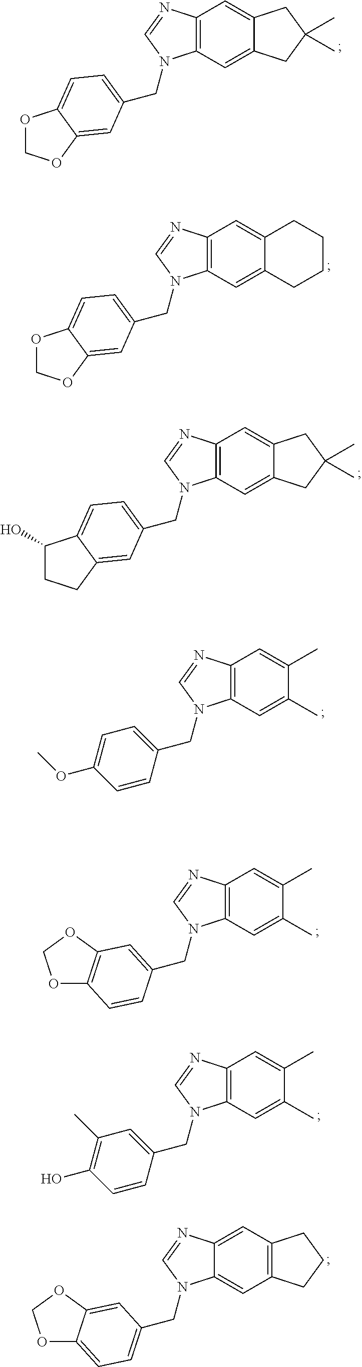 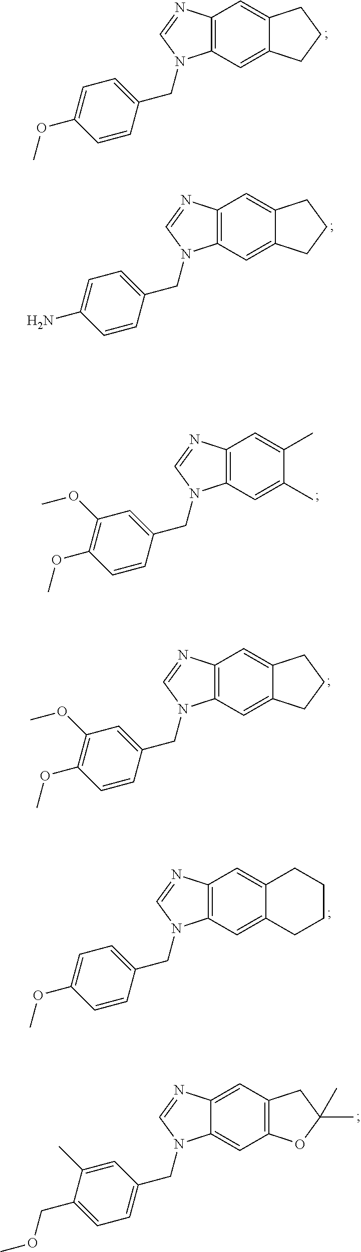 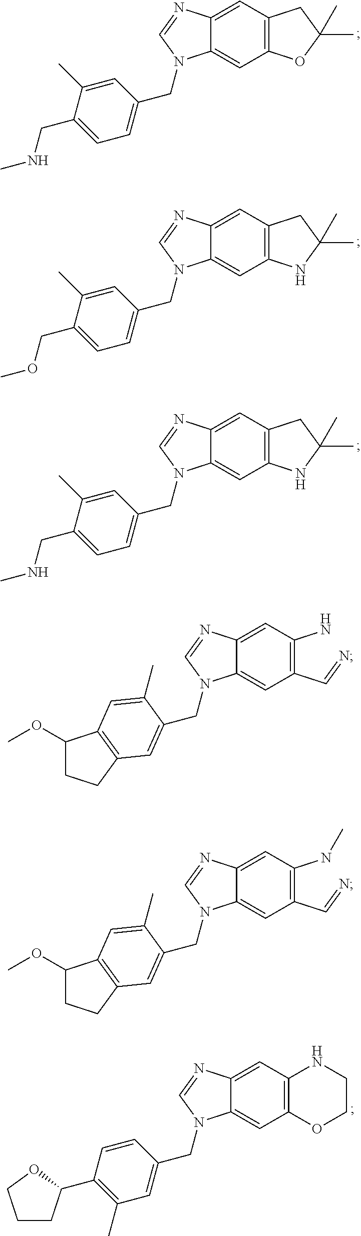 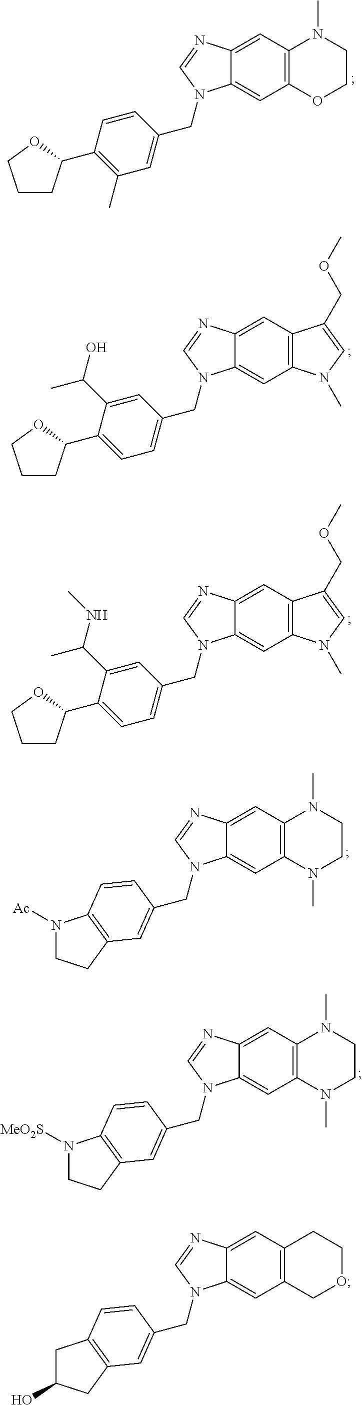 [SMILES: Cc1cc(Cn2cnc3cc4c(cc32)OCCN4C)ccc1[C@@H]1CCCO1 ; COCc1cn(C)c2cc3c(cc12)ncn3Cc1ccc([C@@H]2CCCO2)c(C(C)O)c1 ; CNC(C)c1cc(Cn2cnc3cc4c(COC)cn(C)c4cc32)ccc1[C@@H]1CCCO1 ; CC(=O)N1CCc2cc(Cn3cnc4cc5c(cc43)N(C)CCN5C)ccc21 ; CN1CCN(C)c2cc3c(cc21)ncn3Cc1ccc2c(c1)CCN2S(C)(=O)=O ; O[C@H]1Cc2ccc(Cn3cnc4cc5c(cc43)COCC5)cc2C1] 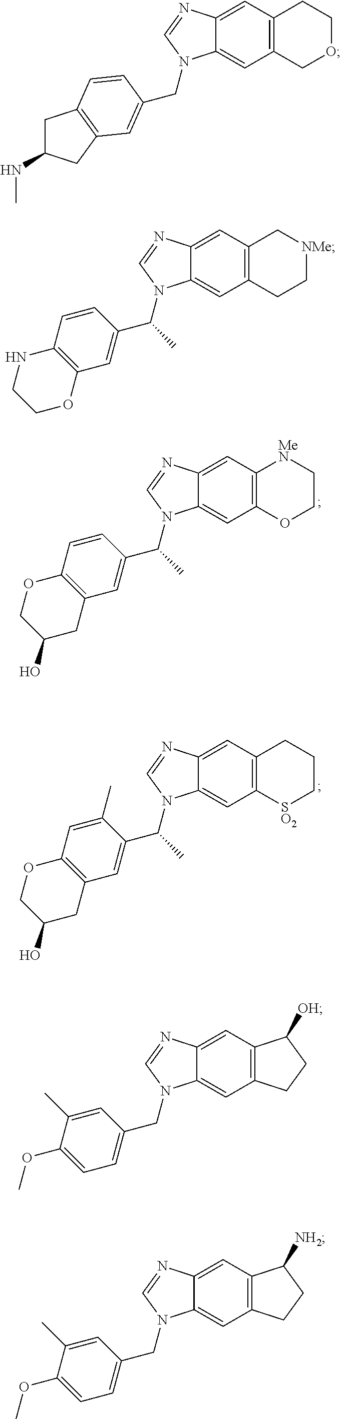 [SMILES: CN[C@H]1Cc2ccc(Cn3cnc4cc5c(cc43)COCC5)cc2C1 ; C[C@H](c1ccc2c(c1)OCCN2)n1cnc2cc3c(cc21)CCN(C)C3 ; C[C@H](c1ccc2c(c1)C[C@@H](O)CO2)n1cnc2cc3c(cc21)OCCN3C ; Cc1cc2c(cc1[C@@H](C)n1cnc3cc4c(cc31)S(=O)(=O)CCC4)C[C@@H](O)CO2 ; COc1ccc(Cn2cnc3cc4c(cc32)CC[C@@H]4O)cc1C ; COc1ccc(Cn2cnc3cc4c(cc32)CC[C@@H]4N)cc1C] 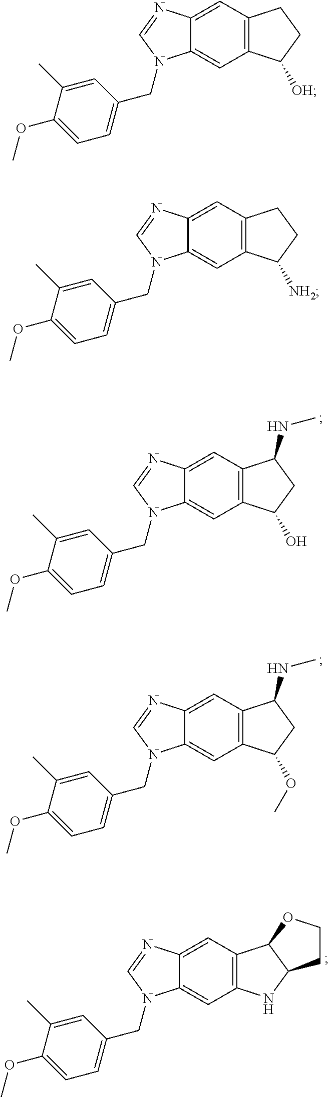 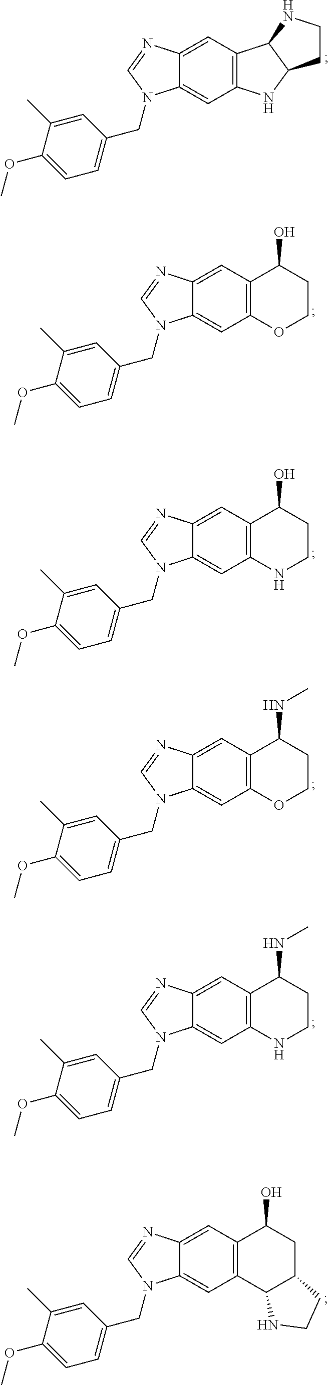 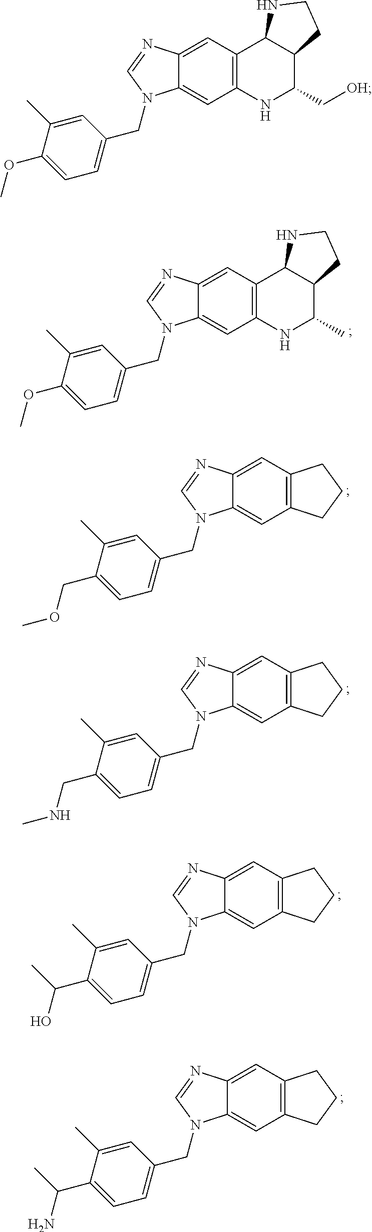 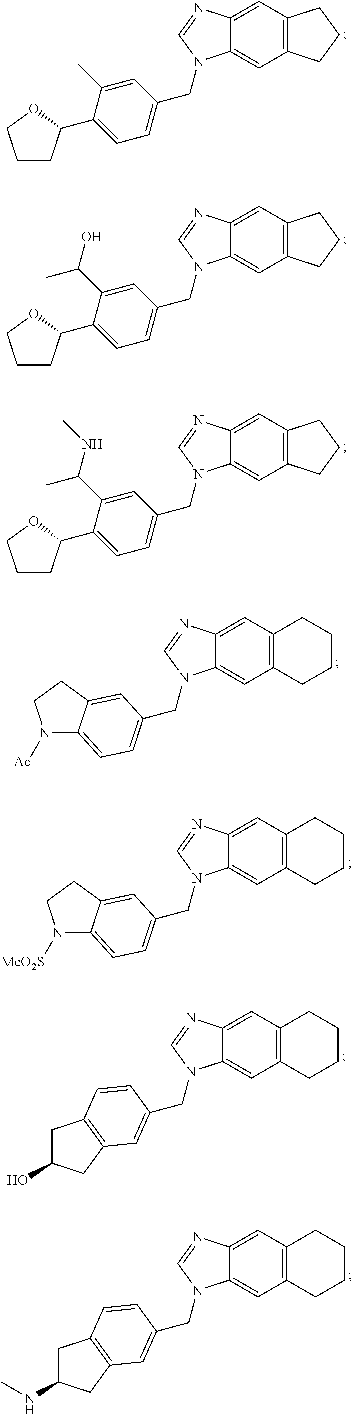 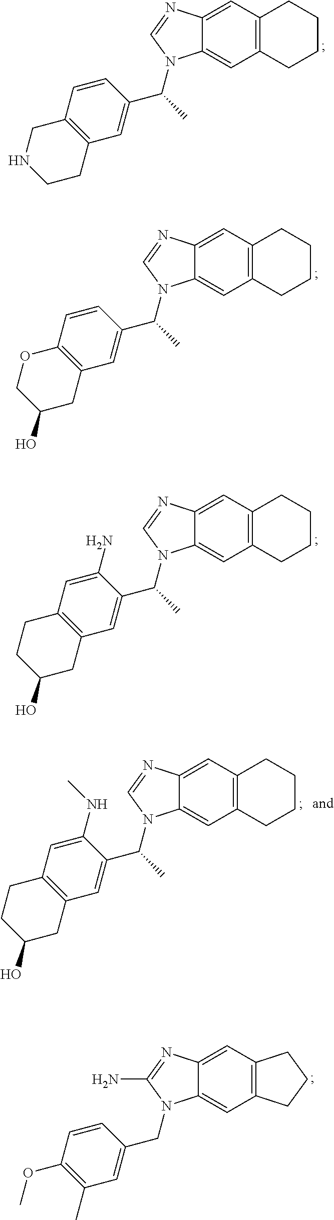 [SMILES: C[C@H](c1ccc2c(c1)CCNC2)n1cnc2cc3c(cc21)CCCC3 ; C[C@H](c1ccc2c(c1)C[C@@H](O)CO2)n1cnc2cc3c(cc21)CCCC3 ; C[C@H](c1cc2c(cc1N)CC[C@H](O)C2)n1cnc2cc3c(cc21)CCCC3 ; CNc1cc2c(cc1[C@@H](C)n1cnc3cc4c(cc31)CCCC4)C[C@@H](O)CC2 ; COc1ccc(Cn2c(N)nc3cc4c(cc32)CCC4)cc1C]
or a pharmaceutically acceptable salt, amide, ester, or prodrug form thereof.

20. A pharmaceutical composition comprising a therapeutically effective amount of a compound according to claim 1 in combination with a pharmaceutically suitable carrier.

21. (canceled)

22. (canceled)

23. (canceled)

24. A method for treating or preventing a bacterial infection comprising administering to a subject in need thereof a therapeutically effective amount of a compound of claim 1.

25. The method of claim 24, wherein the bacterial infection is caused by a bacterium that expresses a FabI protein.

26. The method of claim 24, wherein the bacterial infection is caused by a bacterium selected from the group consisting of Francisella tularensis, Staphylococcus aureus, Bacillus anthracia, Plasmodium falciparum, Yersinia pestis, Enterococcus faecium, Staphylococcus epidermis, Staphylococcus sapropbyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsia rickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia colt Haemophilus influenzae, Helicobacter pylori, Klebsiella pneumoniae, Neisseria meningitidis, Mycobacterium tuberculosis, Plasmodium falciparum, and any combination thereof.

27. The method of claim 24, wherein the bacterial infection is caused by Francisella tularensis.

28. The method of claim 24, wherein bacterial infection is caused by methicillin-resistant Staphylococcus aureus (MRSA), or vancomycin-resistant Staphylococcus aureus (VRSA).

29. The method of claim 27, where the bacterial infection is tularemia.

30. The method of claim 26, where the compound inhibits Enoyl-ACP Reductase (FabI).

31. A method for inhibiting Enoyl-ACP Reductase (FabI), the method comprising contacting Enoyl-ACP Reductase (FabI) with the compound of claim 1.
